(19) United States
(12) Patent Application Publication
(10) Pub. No.: US 2016/0275240 A1
(43) Pub. Date: Sep. 22, 2016

(54) METHODS AND COMPOSITIONS FOR POOLING AMPLIFICATION PRIMERS

(71) Applicant: NuGEN Technologies, Inc., San Carlos, CA (US)

(72) Inventors: Stephanie HUELGA, Belmont, CA (US); Jonathan SCOLNICK, San Francisco, CA (US); Doug AMORESE, Los Altos, CA (US)

(21) Appl. No.: 15/047,448

(22) Filed: Feb. 18, 2016

Related U.S. Application Data
(60) Provisional application No. 62/117,955, filed on Feb. 18, 2015.

(51) Int. Cl. G06F 19/22; C12Q 1/68

(52) U.S. Cl. CPC G06F 19/22; C12Q 1/6869

(57) ABSTRACT

Provided herein are methods, compositions, systems, and kits for pooling amplification primers. Such methods, compositions, systems, and kits can be useful for integrated analysis of multiple classes of genomic alterations in a single assay.

### CROSS-REFERENCE

This patent application claims the benefit of U.S. Provisional Application Ser. No. 62/117,955 filed Feb. 18, 2015, which is incorporated by reference herein in its entirety.

### SEQUENCING LISTING

This application contains a Sequence Listing which is concurrently submitted as an ASCII text file via EFS-Web. The concurrently submitted ASCII text file is named “25115_768_201 SL.txt”, was created on Feb. 18, 2016, and is 2 MB in size. The material in this submitted text file is hereby incorporated by reference in its entirety into this application.

### BACKGROUND

In a clinical sample the specific type of genomic alterations resulting in disease or disorder can be unknown. Classes of genomic alterations can include, by way of example only, single nucleotide polymorphisms, copy number variations, genome re-arrangements, abnormal gene expression, gene fusions, alternative splicing events, or a combination thereof. DNA sequencing can be used to assay such genomic alterations. While whole genome sequencing can be used to assay various classes of genomic alterations, whole genome sequencing can be cost-prohibitive for a large number of clinical and diagnostic applications. Therefore, it can be more practical and cost-effective to select genomic regions of interest for sequencing and analysis. Accordingly, target enrichment can be a commonly employed strategy in genomic sequencing in which genomic regions of interest are selectively captured from a polynucleotide sample before sequencing. However, each class of genomic alterations can require different requirements for target enrichment, sample processing, and data analysis steps. These disparate requirements can present significant challenges to the integrated analysis of multiple classes of genomic alterations in a single sample. Therefore, independent assays can be performed for each class of genomic alterations. For example, targeted sequencing assays can measure only one type of genomic alteration at a time (e.g., SNPs or CNV, but not SNPs and CNV in a single assay). The use of independent assays for each class of genomic alterations can result in increased cost, increased amounts of sample, increased manpower, and increased time. Therefore, there is a need for methods, kits, and systems for integrated analysis of multiple classes of genomic alterations in a single assay.

### SUMMARY

In one aspect, provided herein is a method for detecting presence or absence of two or more classes of genomic alterations in a single assay, the method comprising: (a) sequencing a plurality of polynucleotide library members to produce sequence reads; (b) with aid of a computer processor, querying the sequence reads for presence of a sequence corresponding to any one of a first or second sub-plurality of a plurality of primers, where the first sub-plurality of primers comprises sequence designed to prime extension reactions into target sequence corresponding to genomic locations suspected of harboring a first class of genomic alterations and the second sub-plurality of primers comprises sequence designed to prime extension reactions into target sequence corresponding to genomic locations suspected of harboring a second class of genomic alterations, where the first class of genomic alterations and second class of genomic alterations are different, thereby identifying a first subset of sequence reads generated by sequencing the polynucleotide library members generated using the first sub-plurality of primers and a second subset of sequence reads generated by sequencing the polynucleotide library members generated using the second sub-plurality of primers; (c) with aid of a computer processor, separating the first subset of sequence reads into a first data file, and separating the second subset of sequence reads into a second data file; and (d) with aid of a computer processor, analyzing the first subset of sequence reads for presence or absence of the first class of genomic alterations, and analyzing the second subset of sequence reads for presence or absence of the second class of genomic alterations. In some embodiments, the method comprises, before (a), hybridizing the plurality of primers to a sample of polynucleotides In some embodiments, the method further comprises extending the plurality of primers with a polymerase, thereby generating polynucleotide extension products. In some embodiments, the method further comprises amplifying the polynucleotide extension products, thereby generating amplification products.

In some embodiments, the polynucleotide extension products are the polynucleotide library members of (a). In some embodiments, the amplification products are the polynucleotide library members of (a). In some embodiments, the plurality of primers comprises n additional sub-pluralities of the plurality of primers comprising target-specific sequences designed to extend into target sequence corresponding to genomic locations suspected of harboring n additional classes of genomic alterations. In some embodiments, the sequence reads of (a) further comprise n additional subsets of sequence reads comprising sequences corresponding to the n additional sub-pluralities of the plurality of primers.

In some embodiments, the querying of (b) further comprises querying the sequence reads for presence of a sequence corresponding to the n additional sub-pluralities of the plurality of primers, thereby identifying n additional subsets of sequence reads generated by sequencing the polynucleotide library members generated using the n sub-pluralities of primers. In some embodiments, (c) further comprises separating the n additional subsets of sequence reads into n additional data files. In some embodiments, (d) further comprises analyzing the n additional subsets of sequence reads for presence or absence of the n additional classes of genomic alterations.

In some embodiments, (c) comprises storing the first subset of sequence reads into the first data file, and storing the second subset of sequence reads into the second data file.

In some embodiments, the method further comprises appending a first adaptor sequence to the polynucleotides. In some embodiments, the appending comprises ligation. In some embodiments, the appending comprises appending the first adaptor sequence to a 5′ end of the polynucleotides.

In some embodiments, the plurality of primers comprises a 5′ tail, where the 5′ tail comprises a second adaptor sequence. In some embodiments, the plurality of primers comprises a 3′ portion comprising a target-specific sequence designed to prime an extension reaction into a target sequence corresponding to a genomic location.

In some embodiments, the first adaptor sequence is distinct from the second adaptor sequence. In some embodiments, the first adaptor sequence or the second adaptor sequence comprise a barcode sequence. In some embodiments, the barcode sequence comprises a random hexamer sequence.

In some embodiments, the method further comprises extending the plurality of primers with a polymerase, thereby generating polynucleotide extension products, amplifying the polynucleotide extension products, thereby generating amplification products, where the amplifying comprises use of primers that are at least 70% identical to the first adaptor sequence and the second adaptor sequence.

In some embodiments, the polynucleotides comprise DNA. In some embodiments, the polynucleotides comprise RNA.

In some embodiments, the analyzing comprises simultaneously analyzing the first and second subsets of sequence reads.

In some embodiments, the first class or second class of genomic alterations are selected from the group consisting of single nucleotide polymorphisms (SNPs), insertions, deletions, altered expression levels, gene fusions, copy number variations, copy number alterations, inversions, and translocations.

In some embodiments, a sub-plurality of primers of the plurality of primers is designed to prime an extension reaction into target sequence corresponding to genomic locations suspected of harboring a mutation that results in altered expression levels. In some embodiments, the sub-plurality of the plurality of primers designed to prime an extension reaction into target sequence corresponding to genomic locations suspected of harboring a mutation that results in altered expression levels comprise primers designed to anneal within 5′ and 3′ exons of a gene suspected of having altered expression level. In some embodiments, the sub-plurality of the plurality of primers designed to prime an extension reaction into target sequence corresponding to genomic locations suspected of harboring a mutation that results in altered expression levels comprises primers designed to anneal within 5′ or 3′ exons of a housekeeping gene. In some embodiments, the primers in the sub-plurality of primers designed to prime an extension reaction into target genomic locations suspected of harboring altered expression levels are unique within a transcriptome. In some embodiments, the sub-plurality of the plurality of primers designed to prime an extension reaction into target sequence corresponding to genomic locations suspected of harboring a mutation that results in altered expression levels have a length of at least 35 bases. In some embodiments, the sub-plurality of the plurality of primers designed to prime an extension reaction into target sequence corresponding to genomic locations suspected of harboring a mutation that results in altered expression levels anneal at least 25 bases away from an exon junction.

In some embodiments, a sub-plurality of the plurality of primers is designed to prime an extension reaction into target sequence corresponding to genomic locations suspected of harboring SNPs. In some embodiments, the sub-plurality of the plurality of primers is designed to prime an extension reaction into target sequence corresponding to genomic locations suspected of harboring SNPs are designed to anneal to genomic locations no more than 40 bases away from the SNPs.

In some embodiments, a sub-plurality of the plurality of primers is designed to prime an extension reaction into target sequence corresponding to genomic locations suspected of encoding a transcript that is alternatively spliced. In some embodiments, the primers belonging to the sub-plurality of the plurality of primers designed to prime an extension reaction into target sequence corresponding to genomic locations suspected of encoding a transcript that is alternatively spliced have a length of at least 40 bases. In some embodiments, the sub-plurality of the plurality of primers designed to prime an extension reaction into target sequence corresponding to genomic locations suspected of encoding a transcript that is alternatively spliced comprises primers designed such that the 3′ end of the primers is no more than 25 bases from an exon junction. In some embodiments, the sub-plurality of the plurality of primers designed to prime an extension reaction into target sequence corresponding to genomic locations suspected of encoding a transcript that is alternatively spliced comprises primers designed to anneal to each exon of a gene suspected of encoding a transcript that is alternatively spliced.

In some embodiments, n=1, such that the plurality of primers is designed to prime extension reactions into target sequence corresponding to genomic regions suspected of harboring a total of three classes of genomic alterations. In some embodiments, the first class of genomic alterations is SNPs, the second class of genomic alterations is copy number variations and a third class of genomic alterations is gene fusion events.

In some embodiments, the polynucleotide extension products were extended from polynucleotides comprising a first adaptor sequence. In some embodiments, at least one primer of the plurality of primers comprises a second adaptor sequence. In some embodiments, the second adaptor sequence is located at a 5′ portion of the at least one primer. In some embodiments, a 3′ portion of the at least one primer comprises a target-specific sequence designed to prime an extension reaction into a target sequence corresponding to a genomic location. In some embodiments, the first adaptor sequence is distinct from the second adaptor sequence. In some embodiments, at least one of the first adaptor sequence and second adaptor sequence further comprises a barcode sequence. In some embodiments, the barcode sequence comprises a random hexamer sequence.

In some embodiments, the polynucleotide library members comprise DNA. In some embodiments, the polynucleotide library members comprise RNA. In some embodiments, the polynucleotide library members comprise DNA and RNA.

In some embodiments, the first sub-plurality of primers comprise about one to about one hundred thousand primers. In some embodiments, the plurality of primers are not used to amplify a whole exome.

In some embodiments, the target sequence corresponding to genomic locations are suspected of harboring cancer-related genomic alterations. In some embodiments, the target sequence corresponding to genomic locations are suspected of harboring cardiovascular disease-related genomic alterations. In some embodiments, the target sequence corresponding to genomic locations are suspected of harboring neurological disease-related genomic alterations. In some embodiments, the target sequence corresponding to genomic locations are suspected of harboring autoimmune disease-related genomic alterations.

In some embodiments, (d) comprises simultaneously analyzing the first subset of sequence reads and the second subset of sequence reads.

In some embodiments, the computer processor of (c) is the computer processor of (b). In some embodiments, the computer processor of (c) is the computer processor of (d).

In another aspect, disclosed herein is non-transitory computer readable media comprising computer executable code for detecting presence or absence of two or more classes of genomic alterations in a sample subjected to a single assay, the computer readable medium comprising: (a) a database comprising a set of oligonucleotide sequences corresponding to a set of primers, where the set of oligonucleotide sequences comprises: (i) a first subset of oligonucleotide sequences corresponding to a first subset of primers, where the first subset of primers are designed to prime an extension reaction into target sequence corresponding to genomic locations suspected of harboring a first class of genomic alterations, and (ii) a second subset of oligonucleotide sequences corresponding to a second subset of primers, where the second subset of primers are designed to prime an extension reaction into target sequence corresponding to genomic locations suspected of harboring a second class of genomic alterations; (b) a set of computer executable instructions that, when executed by a processor, performs: (i) receiving a set of sequence reads; (ii) querying the set of sequence reads for presence of a sequence belonging to the first subset of oligonucleotide sequences or second subset of oligonucleotide sequences in the database; (iii) transferring sequence reads which comprise a sequence belonging to the first subset of oligonucleotide sequences into a first data file; (iv) transferring sequence reads which comprise a sequence belonging to the second subset of oligonucleotide sequences into a second data file; and (v) analyzing the sequence reads transferred to the first data file for presence or absence of a first class of genomic alterations, and analyzing the sequence reads transferred to the second data file for presence or absence of a second class of genomic alterations.

In some embodiments, the set of oligonucleotide sequences further comprises n additional subsets of primers, where the n additional subsets of primers are designed to prime an extension reaction into target sequence corresponding to genomic locations suspected of harboring n additional classes of genomic alterations. In some embodiments, the querying further comprises querying the set of sequence reads for presence of a sequence belonging to any one of the n additional subsets of oligonucleotide sequences in the database.

In some embodiments, (iv) further comprises transferring sequence reads which comprise a sequence belonging to at least one of the n additional subsets of oligonucleotide sequences into a corresponding nth additional data file. In some embodiments, (v) further comprises analyzing the sequence reads transferred to the nth additional data files for presence or absence of an nth additional class of genomic alterations. In some embodiments, the analyzing of (v) comprises simultaneously analyzing.

In some embodiments, at least one of the first class, second class, or n additional classes of genomic alterations are selected from the group consisting of single nucleotide polymorphisms (SNPs), insertions, deletions, alternative splicing events, gene fusion events, altered expression levels, copy number variations, copy number alterations, inversions, and translocations.

In some embodiments, the first subset primers comprises about one to about one hundred thousand primers. In some embodiments, the set of primers are not capable of amplifying a whole exome.

In some embodiments, the target sequence corresponding to genomic locations are suspected of harboring cancer-related genomic alterations. In some embodiments, the target sequence corresponding to genomic locations are suspected of harboring cardiovascular disease-related genomic alterations. In some embodiments, the target sequence corresponding to genomic locations are suspected of harboring neurological disease-related genomic alterations. In some embodiments, the target sequence corresponding to genomic locations are suspected of harboring autoimmune disease-related genomic alterations.

In some embodiments, the first subset of primers comprises at least one primer comprising a sequence selected from SEQ ID NOS. 1-12299, and the second subset of primers comprises at least one primer comprising a sequence selected from SEQ ID NOS. 12300-35857.

In some embodiments, (b)(iii) and (b)(iv) are performed simultaneously.

In some embodiments, the database is a data file or a list.

In another aspect, disclosed herein is a computer system for detecting presence or absence of two or more classes of genomic alterations in a sample subjected to a single targeted assay, comprising: (a) a database comprising: (i) a first subset of oligonucleotide sequences corresponding to a first subset of primers, where the first subset of primers are designed to prime an extension reaction into target sequence corresponding to genomic locations suspected of harboring a first class of genomic alterations, and (ii) a second subset of oligonucleotide sequences corresponding to a second subset of primers, where the second subset of primers are designed to prime an extension reaction into target sequence corresponding to genomic locations suspected of harboring a second class of genomic alterations; and (b) a receiver configured to receive a set of sequence reads generated by sequencing a plurality of polynucleotide library members, where the polynucleotide library members were extended using (i) the first subset of primers, and (ii) the second subset of primers; and (c) a processor operatively coupled to the receiver, where the processor comprises computer executable instructions that, when executed by the processor, performs: (i) querying the set of sequence reads for presence of a sequence belonging to the first subset of oligonucleotide sequences or second subset of oligonucleotide sequences in the database; (ii) transferring sequence reads which comprise a sequence belonging to the first subset of oligonucleotide sequences into a first data file; (iii) transferring sequence reads which comprise a sequence belonging to the second subset of oligonucleotide sequences into a second data file; (iv) analyzing the sequence reads transferred to the first data file for presence or absence of a first class of genomic alterations, and analyzing the sequence reads transferred to the second data file for presence or absence of a second class of genomic alterations.

In some embodiments, the single targeted assay is a single targeted sequencing assay.

In some embodiments, (c)(iv) comprises simultaneously analyzing the sequence reads transferred to the first data file and the sequence reads transferred to the second data file.

In some embodiments, the database is a data file or a list.

In another aspect, disclosed herein are kits for detecting presence or absence of two or more classes of genomic alterations in a sample subjected to a single targeted assay, comprising: (a) a plurality of primers, where the plurality of primers comprises (i) a first subset of primers designed to prime an extension reaction into target sequence corresponding to genomic locations suspected of harboring a first class of genomic alterations, and (ii) a second subset of primers designed to prime an extension reaction into target sequence corresponding to genomic locations suspected of harboring a second class of genomic alterations, where the first class of genomic alterations and the second class of genomic alterations are different; (b) a polymerase; and (c) instructions for detecting presence or absence of two or more classes of genomic alterations in a single targeted assay.

In some embodiments, the plurality of primers comprises n additional sub-pluralities of primers comprising target sequence designed to extend into target sequence corresponding to genomic locations suspected of harboring n additional classes of genomic alterations.

In some embodiments, the instructions are instructions for simultaneously detecting presence or absence of two or more classes of genomic alterations in a single targeted assay. In some embodiments, the single targeted assay is a targeted sequencing assay.

In some embodiments, the kit further comprises a non-transitory computer readable medium.

In some embodiments, at least one primer of the first subset comprises an adaptor sequence. In some embodiments, the adaptor sequence is located at a 5′ portion of the at least one primer. In some embodiments, a 3′ portion of the at least one primer comprises a target-specific sequence designed to prime an extension reaction into a target genomic location. In some embodiments, the adaptor sequence further comprises a barcode sequence. In some embodiments, the barcode sequence comprises a random hexamer sequence.

In some embodiments, at least one of the first class, second class or n additional classes of genomic alterations are selected from single nucleotide polymorphisms (SNPs), insertions, deletions, alternative splicing events, gene fusion events, altered expression levels, copy number variations, copy number alterations, inversions, or translocations.

In some embodiments, the first subset of primers is designed to prime an extension reaction into target sequence corresponding to genomic locations suspected of harboring a mutation that result in altered expression levels. In some embodiments, the first subset of primers designed to prime an extension reaction into target sequence corresponding to genomic locations suspected of harboring a mutation that results in altered expression levels comprises primers designed to reside within 5′ or 3′ exons of a gene suspected of having altered expression level. In some embodiments, the first subset of primers designed to prime an extension reaction into target sequence corresponding to genomic locations suspected of harboring a mutation that results in altered expression levels comprises primers designed to reside within 5′ or 3′ exons of a housekeeping gene. In some embodiments, the primers in the first subset of primers designed to prime an extension reaction into target sequence corresponding to genomic locations suspected of harboring mutations that result in altered expression levels are unique. In some embodiments, the first subset of primers designed to prime an extension reaction into target sequence corresponding to genomic locations suspected of harboring a mutation that results in altered expression levels have a length of at least 35 bases. In some embodiments, the first subset of primers designed to prime an extension reaction into target sequence corresponding to genomic locations suspected of harboring a mutation that results in altered expression levels are located at least 25 bases away from an exon junction.

In some embodiments, the first subset of primers is designed to prime an extension reaction into target sequence corresponding to genomic locations suspected of harboring SNPs. In some embodiments, the first subset of primers is designed to prime an extension reaction into target sequence corresponding to genomic locations suspected of harboring SNPs are designed to anneal to genomic locations no more than 40 bases away from the SNPs.

In some embodiments, the first subset of primers is designed to prime an extension reaction into target sequence corresponding to genomic locations suspected of encoding a transcript that is alternatively spliced. In some embodiments, the first subset of primers belonging to the sub-plurality of primers designed to prime an extension reaction into target sequence corresponding to genomic locations suspected of encoding a transcript that is alternatively spliced have a length of at least 40 bases. In some embodiments, the first subset of primers designed to prime an extension reaction into target sequence corresponding to genomic locations suspected of encoding a transcript that is alternatively spliced comprises primers designed such that the 3′ end of the primers anneal no more than 25 bases from an exon junction. In some embodiments, the first subset of primers designed to prime an extension reaction into target sequence corresponding to genomic locations suspected of encoding a transcript that is alternatively spliced comprises primers designed to anneal to each exon of a gene suspected of encoding a transcript that is alternatively spliced.

In some embodiments, n=1, such that the plurality of primers is designed to prime extension reactions into target sequence corresponding to genomic regions suspected of harboring a total of three classes of genomic alterations. In some embodiments, the first class of genomic alterations comprises SNPs, the second class of genomic alterations comprises copy number variations and a third class of genomic alterations comprises gene fusion events.

In some embodiments, the first subset of primers comprises about one to about one hundred thousand primers. In some embodiments, the plurality of primers is not capable of amplifying a whole exome.

In some embodiments, the target sequence corresponding to genomic locations are suspected of harboring cancer-related genomic alterations. In some embodiments, the target sequence corresponding to genomic locations are suspected of harboring cardiovascular disease-related genomic alterations. In some embodiments, the target sequence corresponding to genomic locations are suspected of harboring neurological disease-related genomic alterations. In some embodiments, the target sequence corresponding to genomic locations are suspected of harboring autoimmune disease-related genomic alterations.

In some embodiments, the first subset of primers comprises at least one primer comprising a sequence selected from SEQ ID NOS. 1-12299, and the second subset of primers comprises at least one primer comprising a sequence selected from SEQ ID NOS. 12300-35857.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features described herein are set forth with particularity in the appended claims. A better understanding of the features and advantages described herein will be obtained by reference to the following detailed description that sets forth illustrative embodiments, in which the principles described herein are utilized, and the accompanying drawings of which:

FIG. 1 depicts an exemplary workflow of a method described herein.

FIG. 2 depicts an exemplary embodiment of selective target enrichment method.

FIG. 3 depicts an exemplary computer system described herein.

### DETAILED DESCRIPTION

Methods described herein can employ, unless otherwise indicated, techniques of molecular biology, microbiology and recombinant DNA techniques, which are within the skill of the art. Such
techniques are explained fully in the literature. See, e.g., Sambrook, Fritsch & Maniatis, Molecular Cloning: A Laboratory Manual, Fourth Edition (2012); Oligonucleotide Synthesis (M. J. Gait, ed., 1984); Nucleic Acid Hybridization (B. D. Hames & S. J. Higgins, eds., 1984); A Practical Guide to Molecular Cloning (B. Perbal, 1984); and a series, Methods in Enzymology (Academic Press, Inc.), which are hereby incorporated by reference.

### DEFINITIONS

As used in the specification and claims, the singular forms “a”, “an” and “the” can include plural references unless the context clearly dictates otherwise. For example, the term “a cell” can include a plurality of cells, including mixtures thereof.

“Nucleotides” and “nt” can be used interchangeably herein to refer to biological molecules that can form nucleic acids. Nucleotides can have moieties that contain not only the known purine and pyrimidine bases, but also other heterocyclic bases that have been modified. Such modifications include methylated purines or pyrimidines, acylated purines or pyrimidines, alkylated riboses, or other heterocycles. In addition, the term “nucleotide” can include those moieties that contain hapten, biotin, or fluorescent labels and may contain not only conventional ribose and deoxyribose sugars, but other sugars as well. Modified nucleosides or nucleotides can also include modifications on the sugar moiety, e.g., wherein one or more of the hydroxyl groups are replaced with halogen atoms or aliphatic groups, are functionalized as ethers, amines, or the like. Modified nucleosides or nucleotides can also include peptide nucleic acid (PNA).

The terms “polynucleotides”, “nucleic acid”, “nucleotides” and “oligonucleotides” can be used interchangeably. They can refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, transfer-messenger RNA, ribosomal RNA, antisense RNA, small nuclear RNA (snRNA), small nucleolar RNA (snoRNA), micro-RNA (miRNA), small interfering RNA (siRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. A nucleic acid described herein can contain phosphodiester bonds, although in some cases, as outlined below (for example in the construction of primers and probes such as label probes), nucleic acid analogs are included that can have alternate backbones, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid (also referred to herein as “PNA”) backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with bicyclic structures including locked nucleic acids (also referred to herein as “LNA”), Koshkin et al., J. Am. Chem. Soc. 120.13252 3 (1998); positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp 169 176). Several nucleic acid analogs are described in Rawls, C & E News Jun. 2, 1997 page 35. “Locked nucleic acids” are also included within the definition of nucleic acid analogs. LNAs can be a class of nucleic acid analogues in which the ribose ring is “locked” by a methylene bridge connecting the 2′-0 atom with the 4′-C atom. All of these references are hereby expressly incorporated by reference. These modifications of the ribose-phosphate backbone can be done to increase the stability and half-life of such molecules in physiological environments. For example, PNA:DNA and LNA-DNA hybrids can exhibit higher stability and thus can be used in some embodiments. The target nucleic acids can be single stranded or double stranded, as specified, or contain portions of both double stranded or single stranded sequence. Depending on the application, the nucleic acids can be DNA (including, e.g., genomic DNA, mitochondrial DNA, and cDNA), RNA (including, e.g., mRNA and rRNA) or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, etc.

The term “target polynucleotide,”, “target region”, or “target”, as use herein, can refer to a polynucleotide of interest. In certain embodiments, a target polynucleotide can be under study. In certain embodiments, a target polynucleotide contains one or more sequences that are of interest and under study. A target polynucleotide can comprise, for example, a genomic sequence. A target polynucleotide can also comprise extranuclear nucleic acids, e.g., mitochondrial DNA, chloroplast DNA and the like. The target polynucleotide can comprise a target sequence whose presence, amount, and/or nucleotide sequence, or genomic alterations in these, are desired to be determined.

The term “genomic sequence”, as used herein, can refer to a sequence that occurs in a genome. Because RNAs are transcribed from a genome, this term encompasses sequence that exist in the nuclear genome of an organism, as well as sequences that can be present in a cDNA copy of an RNA (e.g., an mRNA) transcribed from such a genome.

The terms “anneal”, “hybridize” or “bind,” can be used interchangeably herein to refer to the combining of one or more single-stranded polynucleotide sequences, segments or strands, and allowing them to form a double-stranded molecule through base pairing. Two complementary sequences (e.g., DNA and/or RNA) can anneal or hybridize by forming hydrogen bonds with complementary bases to produce a double-stranded polynucleotide or a double-stranded region of a polynucleotide.

As used herein, a sequence can “correspond” to a sub-plurality of primers if it is substantially identical or complementary to an identifying sequence of a primer belonging to the sub-plurality of primers. The identifying sequence of a primer can comprise a unique target-selective sequence of the primer that is, e.g., not found in any other primer. The identifying sequence of a primer can be a barcode sequence that is common to all primers in a given sub-plurality of primers in a set, and which is not found in primers belonging to any other sub-plurality of primers in the set. The identifying sequence can be an entire sequence of a primer. In other embodiments, the identifying sequence can be less than the entire sequence of a primer.

As used herein, the term “complementary” can refer to a relationship between one or more antiparallel nucleic acid sequences in which the sequences are related by the base-pairing rules: A can pair with T or U and C can pair with G. A first sequence or segment that is “perfectly complementary” to a second sequence or segment is complementary across its entire length and has no mismatches. A first sequence or segment can be “substantially complementary” to a second sequence of segment when a polynucleotide consisting of the first sequence is sufficiently complementary to specifically anneal to a polynucleotide consisting of the second sequence.

As used herein, “amplification” of a nucleic acid sequence can refer to in vitro techniques for enzymatically increasing the number of copies of a target sequence. Amplification methods can include both methods in which the predominant product is single-stranded and methods in which the predominant product is double-stranded). A “round” or “cycle” of amplification can refer to a polymerase chain reaction (PCR) cycle in which a double stranded template can be denatured into single-stranded templates, forward and reverse primers can be annealed to the single stranded templates to form primer/template duplexes, primers can be extended by a polymerase from the primer/template duplexes to form extension products. In subsequent rounds of amplification the extension products can be denatured into single stranded templates and the cycle can be repeated. Amplification can include PCR. Examples of PCR techniques that can be used in the methods provided herein include quantitative PCR, quantitative fluorescent PCR (QF-PCR), multiplex fluorescent PCR (MF-PCR), real time PCR(RT-PCR), single cell PCR, restriction fragment length polymorphism PCR (PCR-RFLP), PCR-RFLP/RT-PCR-RFLP, hot start PCR, nested PCR, in situ polony PCR, in situ rolling circle amplification (RCA), bridge PCR, picotiter PCR, digital PCR (dPCR), droplet digital PCR (ddPCR), and emulsion PCR. Other suitable amplification methods include the ligase chain reaction (LCR), transcription amplification, molecular inversion probe (MIP) PCR, self-sustained sequence replication, selective amplification of target polynucleotide sequences, consensus sequence primed polymerase chain reaction (CP-PCR), arbitrarily primed polymerase chain reaction (AP-PCR), degenerate oligonucleotide-primed PCR (DOP-PCR) and nucleic acid based sequence amplification (NABSA). Other amplification methods that can be used herein include those described in U.S. Pat. Nos. 5,242,794; 5,494,810; 4,988,617; and 6,582,938. Amplification of target nucleic acids can occur on a bead. In other embodiments, amplification does not occur on a bead. Amplification can be by isothermal amplification, e.g., isothermal linear amplification. A hot start PCR can be performed wherein the reaction is heated to 95° C. e.g., for two minutes prior to addition of a polymerase or the polymerase can be kept inactive until a first heating step in cycle 1. Hot start PCR can be used to minimize nonspecific amplification.

The term “polymerase” as used herein can refer to an enzyme that links individual nucleotides together into a strand, using another strand as a template. Examples of polymerases can include a DNA polymerase, an RNA polymerase, a thermostable polymerase, a wild-type polymerase, a modified polymerase, E. coli DNA polymerase I, T7 DNA polymerase, bacteriophage T4 DNA polymerase Φ29 (phi29) DNA polymerase, Taq polymerase, Tth polymerase, Tli polymerase, Pfu polymerase VENT polymerase, DEEPVENT polymerase, EX-Taq polymerase, LA-Taq polymerase, Sso polymerase, Poc polymerase, Pab polymerase, Mth polymerase, ES4 polymerase, Tru polymerase, Tac polymerase, Tne polymerase, Tma polymerase, Tca polymerase, Tih polymerase, Tfi polymerase, Platinum Taq polymerases, Tbr polymerase, Tfl polymerase, Tth polymerase, Pfutubo polymerase, Pyrobest polymerase, Pwo polymerase, KOD polymerase, Bst polymerase, Sac polymerase, Klenow fragment, polymerase with 3′ to 5′ exonuclease activity, and variants, modified products and derivatives thereof. In some embodiments, the polymerase is a single subunit polymerase. The polymerase can have high processivity, namely the capability of the polymerase to consecutively incorporate nucleotides into a nucleic acid template without releasing the nucleic acid template.

The terms “template,” “template strand,” “template DNA” and “template nucleic acid” can be used interchangeably herein to refer to a strand of DNA or RNA that can copied by an amplification cycle.

The term “denaturing,” as used herein, can refer to the at least partial separation of a nucleic acid duplex into two single strands.

The term “extending”, as used herein, can refer to the extension of a primer annealed to a template nucleic acid by the addition of nucleotides using an enzyme, e.g., a polymerase.

The terms “primer” and “probe” can refer to a nucleotide sequence (e.g., an oligonucleotide) that anneals with a template sequence (such as a target polynucleotide, or a primer extension product). In some instances, a nucleotide sequence can have a free 3′-OH group and can be capable of promoting polymerization of a polynucleotide complementary to the template. A primer can be, for example, a sequence of the template (such as a primer extension product or a fragment of the template created following RNase cleavage of a template-DNA complex) that can be annealed to a sequence in the template itself (for example, as a hairpin loop), and can promote nucleotide polymerization. Thus, a primer can be an exogenous (e.g., added) primer or an endogenous (e.g., template fragment) primer. A primer can be a tailed primer; a tail can comprise a sequence that does not anneal to a template. A tail sequence of a tailed primer can be unhybridized to another sequence. A primer can comprise a 3′ end (e.g., at least 10 nt, 20 nt, 25 nt, 30 nt, 35 nt, 40 nt, 45 nt in length) that anneals to a template and, optionally, a 5′ tail (e.g., at least 10 nt, 20 nt, 25 nt, 30 nt, 35 nt, 40 nt, 45 nt in length) that does not anneal to a template. A 5′ tail can comprise an adaptor sequence.

The terms “determining”, “measuring”, “evaluating”, “assessing,” “assaying,” and “analyzing” can be used interchangeably herein to refer to any form of measurement, and include determining if an element is present or not. These terms can include both quantitative and/or qualitative determinations. Assessing can be relative or absolute. “Assessing the presence of” can include determining the amount of something present, as well as determining whether it is present or absent.

The term “about” as used herein, when referring to a numerical value or range, can allow for a degree of variability of +/−15% of a stated value or of a stated limit of a range.

The term “single nucleotide polymorphism”, or “SNP”, as used herein, can refer to a type of genomic sequence variation resulting from a single nucleotide substitution within a sequence. “SNP alleles” or “alleles of a SNP” can refer to alternative forms of the SNP at particular locus. The term “interrogated SNP allele” can refer to the SNP allele that an assay is designed to detect.

The term “copy number alteration” or “CNA” can refer to differences in the copy number of genetic information. CNA can refer to differences in the per genome copy number of a genomic region. For example, in a diploid organism the expected copy number for autosomal genomic regions is 2 copies per genome. Such genomic regions should be present at 2 copies per cell. CNA can be a source of genetic diversity in humans and can be associated with complex disorders and disease, for example, by altering gene dosage, gene disruption, or gene fusion. A CNA can also represent benign polymorphic variants. CNAs can be large, for example, larger than 1 Mb, or can be smaller, for example between about 1 base and about 1 Mb. In some instances, CNA's between 1 base and 1 kb can be referred to as an “insertion” (if the alteration is an addition) or a “deletion” (if the alteration is a deletion). CNA's can be referred to as “copy number variations” (CNV's). For a review see Zhang et al. Annu. Rev. Genomics Hum. Genet. 2009. 10:451-81. More than 38,000 CNAs greater than 100 bases (and less than 3 Mb) have been reported in humans. Along with SNPs these CNAs can account for a significant amount of phenotypic variation between individuals. In addition to having deleterious impacts, e.g. causing disease, they may also result in advantageous variation.

“Parsing” or “binning” as used herein can refer to a process of arranging or separating a plurality of sequence reads into distinct groups, or bins. Parsed or binned reads can be analyzed by individual data workflows with the aid of a computer processor. For example, sequence reads corresponding to genomic targets suspected of harboring SNPs can be binned or parsed based on a sequence of a probe (primer) designed to prime extension reactions into target sequence corresponding to genomic locations suspected of harboring SNPs.

### Overview

Described herein are methods, compositions, kits, and systems for integrated analysis of multiple classes of genomic alterations in a single assay. Methods described herein can improve genomic analysis by reducing the computational burden for analysis of multiple types of genomic alterations from a single experiment. Methods described herein can improve genomic analysis by reducing the time required for completing the assay and analysis from start to finish. Methods described herein can improve genomic analysis by enabling simultaneous detection of multiple classes of genomic alterations in a single sample.

A method described herein can comprise subjecting a sample of polynucleotides to target enrichment. Target enrichment can be carried out using a plurality of primers designed to amplify target sequences corresponding to genomic regions of interest. For example, a target sequence corresponding to a genomic region can be the same as a genomic region. In some instances, a target sequence corresponding to a genomic region is a transcript of a genomic region. In some instances, a target sequence is an exon. In some instances, a target sequence is an intron. In some instances, a target sequence is an exon/intron boundary. A target sequence can be, e.g., a non-repetitive sequence (e.g., a protein coding gene or an RNA-coding gene), or a repetitive sequence (e.g., tandem repeat, interspersed repeat, retrotransposon (e.g., long terminal repeat (LTR), non-long terminal repeat (Non-LTR), long interspersed element (LINEs), short interspersed elements (SINES), or DNA transposons. The target sequences corresponding to genomic regions of interest can be suspected of harboring one or a plurality of classes of genomic alterations. The plurality of primers can comprise multiple sub-pluralities of primers, wherein each sub-plurality is designed to amplify target regions suspected of harboring a specific class of genomic alterations. The target-enriched sample can be pre-amplified prior to sequencing. The target-enriched sample can then be sequenced to produce sequence reads. The sequence reads can then be queried for presence of a sequence corresponding to any primer sequence in any one of the sub-pluralities of primers. Sequence reads can be sorted into separate data files according to presence of a sequence corresponding to a particular sub-plurality of primers. Sequence reads in particular data files can be separated with the aid of a computer processor for detection of a particular class of genomic alterations.

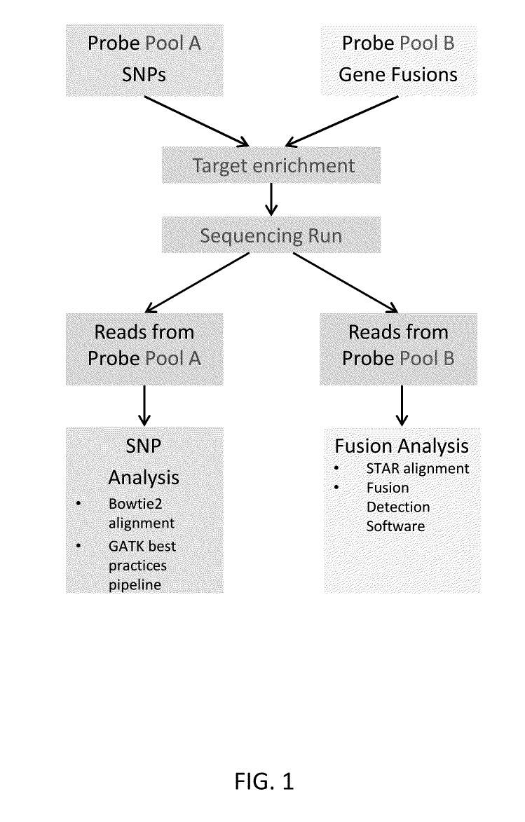

FIG. 1 depicts an exemplary embodiment of a method described herein. A plurality of primers comprising two sub-pluralities of primers (“Probe Pool A” and “Probe Pool B”) is prepared. Probe Pool A comprises primers designed to prime extension reactions into target sequences corresponding to genomic locations suspected of harboring SNPs (e.g., primers SEQ ID NOs: 1-12,299, incorporated by reference from the text file named “25115_768_201 SL.txt”, was created on Feb. 18, 2016). Probe Pool B comprises primers designed to prime extension reactions into target sequences corresponding to genomic locations suspected of harboring gene fusions (e.g., primers SEQ ID NOs: 12,300-35857, incorporated by reference from the text file is named “25115_768_201 SL.txt”, was created on Feb. 18, 2016). Probes from Pools A and B are pooled together and used for target enrichment of polynucleotides in a biological sample. The target-enriched sample is then subjected to sequencing library preparation as described herein or otherwise known in the art. The resulting target-enriched library is subjected to sequencing, thereby producing sequence reads. The sequence reads are queried for presence of any primer sequence corresponding to Probe Pool A or B. Sequence reads comprising primer sequences corresponding to Probe Pool A are subjected to a SNP analysis bioinformatics workflow. Exemplary SNP analysis workflows include, e.g., Bowtie analysis and Genome Analysis toolkit (GATK) best practices pipeline. Sequence reads comprising primer sequences corresponding to Probe Pool B are subjected to a gene fusion analysis bioinformatics workflow. Exemplary gene fusion analysis bioinformatics workflows include, e.g., Spliced Transcripts Alignment to a Reference (STAR) alignment or other fusion detection software. The SNP analysis and gene fusion analysis can be performed simultaneously.

Exemplary Samples

The sample of polynucleotides can be obtained from a biological sample obtained from a subject. The biological sample can be a solid biological sample, e.g., a tumor sample. In some embodiments, a sample from a subject can comprise at least 1%, 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% tumor cells or nucleic acid from a tumor. The solid biological sample can be processed by fixation in a formalin solution, followed by embedding in paraffin (e.g., a FFPE sample). The solid biological sample can be processed by freezing. Alternatively, the biological sample can be neither fixed nor frozen. The unfixed, unfrozen sample can be stored in a solution configured for the preservation of nucleic acid. The solid biological sample can optionally be subjected to homogenization, sonication, French press, dounce, freeze/thaw, which can be followed by centrifugation.

The sample can be a liquid biological sample. For example, the liquid biological sample can be a blood sample (e.g., whole blood, plasma, or serum). A whole blood sample can be subjected to separation of a cellular components (e.g., plasma, serum) and cellular components by use of a Ficoll reagent. In some embodiments, the liquid biological sample can be a urine sample. In some embodiments, the liquid biological sample can be a perilymph sample. In some embodiments, the liquid biological sample can be a fecal sample. In some embodiments, the liquid biological sample can be saliva. In some embodiments, the liquid biological sample can be semen. In some embodiments, the liquid biological sample can be amniotic fluid. In some embodiments, the liquid biological sample can be cerebrospinal fluid. In some embodiments, the liquid biological sample can be bile. In some embodiments, the liquid biological sample can be sweat. In some embodiments, the liquid biological sample can be tears. In some embodiments, the liquid biological sample can be sputum. In some embodiments, the liquid biological sample can be synovial fluid. In some embodiments, the liquid biological sample can be vomit. In some embodiments, the liquid biological sample can be a cell-free sample. In some specific embodiments, the cell-free sample can be a cell-free plasma sample.

Polynucleotides in a sample (which can be referred to as input nucleic acid or input) can comprise DNA. The input nucleic acid can be complex DNA, such as double-stranded DNA, genomic DNA or mixed nucleic acids from more than one organism. In some instances, an input nucleic acid sample can comprise a mixture of nucleic acids from a human and a microbe. In some instances, an input nucleic acid sample can comprise a mixture of nucleic acids from a human and a pathogen. In some instances, an input nucleic acid sample can comprise a mixture of nucleic acids from a human and a virus. In some instances, an input nucleic acid sample can comprise a mixture of nucleic acids from a human and a parasite. In some instances, an input nucleic acid sample can comprise a mixture of nucleic acids from a human and a fetus.

Polynucleotides in the sample can comprise RNA. The RNA can be obtained and purified. RNA can include RNAs in purified or unpurified form, which include, but are not limited to, mRNAs, tRNAs, snRNAs, rRNAs, retroviruses, small non-coding RNAs, microRNAs, polysomal RNAs, pre-mRNAs, intronic RNA, viral RNA, cell free RNA and fragments thereof. The non-coding RNA, or ncRNA may include snoRNAs, microRNAs, siRNAs, piRNAs and long nc RNAs. Polynucleotides in the sample can comprise cDNA. The cDNA can be generated from RNA, e.g., mRNA. The cDNA can be single or double stranded. Polynucleotides in the sample can be of a specific species, for example, human, rat, mouse, other animals, specific plants, bacteria, algae, viruses, and the like. Polynucleotides in the sample can be from a mixture of genomes of different species such as host-pathogen, bacterial populations, and the like. For example, the input DNA can be cDNA made from a mixture of genomes of different species. Alternatively, the input nucleic acid can be from a synthetic source. The input DNA can be mitochondrial DNA. The input DNA can be cell-free DNA. The cell-free DNA can be obtained from, e.g., a serum or plasma sample. The input DNA can comprise one or more chromosomes. For example, if the input DNA is from a human, the DNA can comprise one or more of chromosome 1, 2, 3, 4, 5, 6, 7, 8, 9, 10, 11, 12, 13, 14, 15, 16, 17, 18, 19, 20, 21, 22, X, or Y. The DNA can be from a linear or circular genome. The DNA can be plasmid DNA, cosmid DNA, bacterial artificial chromosome (BAC), or yeast artificial chromosome (YAC). The input DNA can be from more than one individual or organism. The input DNA can be double stranded or single stranded. The input DNA can be part of chromatin. The input DNA can be associated with histones.

A nucleic acid may have a natural or artificial structure, or a combination thereof. Nucleic acids with a natural structure, namely, deoxyribonucleic acid (DNA) and ribonucleic acid (RNA), generally have a backbone of alternating pentose sugar groups and phosphate groups. Each pentose group can be linked to a nucleobase (e.g., a purine (such as adenine (A) or guanine (T)) or a pyrimidine (such as cytosine (C), thymine (T), or uracil (U))). Nucleic acids with an artificial structure can be analogs of natural nucleic acids and may, for example, be created by changes to the pentose and/or phosphate groups of the natural backbone. Exemplary artificial nucleic acids include glycol nucleic acids (GNA), peptide nucleic acids (PNA), locked nucleic acid (LNA), threose nucleic acids (TNA), and the like.

In some instances, a plurality of samples can be from the same subject. In some instances, a plurality of samples can be from a plurality of different subjects. In some embodiments, a plurality of samples can be from at least 1, 2, 3, 4, 5, 6, 7, 8, 9, 10, 20, 30, 40, 50, 60, 70, 80, 90, 100, 150, 200, 250, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, 9500 or 10000 subjects.

In some embodiments, samples can be collected over a period of time. Samples can be collected over regular time intervals, or can be collected intermittently over irregular time intervals. In some instances, a sample can be collected at least every 1, 5, 10, 20, 30, 45 or 60 minutes. In some instances, a sample can be collected at least every 1, 2, 3, 4, 5, 6, 7, 8, 9, 10, 11, 12, 13, 14, 15, 16, 17, 18, 19, 20, 21, 22, 23, 24, 36, 48, 72 or 96 hours. In some instances, a sample can be collected at least every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 days. In some instances, a sample can be collected at least every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 months. In some embodiments, a sample can be collected at least every 0.5, 1, 1.5, 2, 2.5, 3, 3.5, 4, 4.5, 5, 5.5, 6, 6.5, 7, 7.5, 8, 8.5, 9, 9.5 or 10 years. Nucleic acids from different samples can be compared, e.g., to monitor a progression or recurrence of a condition, e.g., a disease.

In some instances, a sample can be collected by core biopsy. In some embodiments, a sample can be collected by aspiration, e.g. through the use of a needle and syringe. Examples of samples amenable to aspiration can include a sample or bodily fluid, a cell suspension, or a liquid in a crime scene as exemplary examples. In some instances, a sample can be collected by scraping. Examples of scraping can include swabbing a patients cheek or scrubbing a crime scene as exemplary examples. In some instances, a sample can be collected through excavation. In some embodiments, a sample can be collected as a purified nucleic acid. Examples of such purified samples can include precipitated nucleic acid affixed to filter paper, phenol-chloroform extractions, nucleic acid purified by kit purification (e.g. Quigen Miniprep™ and the like), or gel purified nucleic acid as exemplary examples.

In some embodiments, a sample can be provided directly from a patient. In some embodiments, a sample can be provided indirectly from a patient through a third party. In some embodiments, a sample can come from a crime scene. In some embodiments, a sample can come from a public water supply. In some embodiments, a sample can come from an archeological excursion. In some embodiments, a sample can be collected for forensic analysis. In some embodiments, a sample can be collected for a security application, e.g., detection of a bioterrorism agent. In some embodiments, samples are collected for determining genealogical connections, e.g., samples taken from relatives (child, brother, sister, mother, father, aunt, uncle, grandfather, great grandfather, grandmother, great grandmother, or cousin). A sample can comprise a historical sample, e.g., a historical FFPE sample.

Exemplary Sample Preparation

Sample preparation can comprise fragmenting polynucleotides in an input sample to generate polynucleotide fragments. The nucleic acids can be e.g. DNA, or RNA. The nucleic acids can be single or double stranded. The DNA can be genomic DNA, extranuclear DNA (e.g. mitochondrial DNA), cDNA or any combination thereof. The nucleic acids in an input sample can be single or double stranded DNA. Fragmentation of the nucleic acids can be achieved. Fragmentation can be through physical fragmentation methods and/or enzymatic fragmentation methods. Physical fragmentation methods can include nebulization, sonication, and/or hydrodynamic shearing. Fragmentation can be accomplished mechanically comprising subjecting the nucleic acids in the input sample to acoustic sonication. Fragmentation can comprise treating the nucleic acids in the input sample with one or more enzymes under conditions suitable for the one or more enzymes to generate double-stranded nucleic acid breaks. Examples of enzymes useful in the generation of nucleic acid or polynucleotide fragments include sequence specific and non-sequence specific nucleases. Non-limiting examples of nucleases can include DNase I, RNase A, Fragmentase, Serratia marcescens endonuclease, restriction endonucleases, variants thereof, and combinations thereof. Reagents for carrying out enzymatic fragmentation reactions can be commercially available (e.g., from New England Biolabs). As a non-limiting example, digestion with DNase I can induce random double-stranded breaks in DNA in the absence of Mg²⁺ and in the presence of Mn²⁺. Fragmentation can comprise treating the nucleic acids in the input sample with one or more restriction endonucleases. Fragments can be generated using, e.g., a clustered regularly-interspaced short palidromic repeats (CRISPR)/Cas9, using guide RNA (gRNA). Fragments can be generated using zinc finger nucleases. Fragmentation can produce fragments having 5′ overhangs, 3′ overhangs, blunt ends, or a combination thereof. In some situations wherein fragmentation comprises the use of one or more restriction endonucleases, cleavage of sample polynucleotides can leave overhangs having a predictable sequence. Methods provided herein can include a step of size selecting fragments via methods such as column purification or isolation from an agarose gel.

Nucleic acids in an input sample can be fragmented into a population of fragmented nucleic acid molecules or polynucleotides of one or more specific size range(s). The fragments can have an average length from about 10 to about 10,000 nucleotides. The fragments can have an average length from about 50 to about 2,000 nucleotides. The fragments can have an average length from about 100 to about 2,500, about 10 to about 1,000, about 10 to about 800, about 10 to about 500, about 50 to about 500, about 50 to about 250, or about 50 to about 150 nucleotides. The fragments can have an average length less than 10,000 nucleotide, such as less than 5,000 nucleotides, less than 2,500 nucleotides, less than 2,500 nucleotides, less than 1,000 nucleotides, less than 500 nucleotides, such as less than 400 nucleotides, less than 300 nucleotides, less than 200 nucleotides, or less than 150 nucleotides.

Fragmentation of the nucleic acids can be followed by end repair of the nucleic acid fragments. End repair can include the generation of blunt ends, non-blunt ends (e.g., sticky or cohesive ends), or single base overhangs such as the addition of a single dA nucleotide to the 3′-end of the nucleic acid fragments, by a polymerase lacking 3′-exonuclease activity. End repair can be performed using any number of enzymes and/or methods including commercially available kits such as the Encore™ Ultra Low Input NGS Library System I. End repair can be performed on double stranded DNA fragments to produce blunt ends wherein the double stranded DNA fragments contain 5′ phosphates and 3′ hydroxyls. The double-stranded DNA fragments can be blunt-end polished (or “end repaired”) to produce DNA fragments having blunt ends. In some instances, a DNA fragment can be joined to adapters after end repair. Generation of the blunt ends on the double stranded fragments can be generated by the use of a single strand specific DNA exonuclease such as for example exonuclease 1, exonuclease 7 or a combination thereof to degrade overhanging single stranded ends of the double stranded products. Alternatively, the double stranded DNA fragments can be blunt ended by the use of a single stranded specific DNA endonuclease, for example, but not limited to, mung bean endonuclease or S1 endonuclease. Fragments, e.g., fragments generated by amplification with chimeric DNA/RNA primers, can be treated with, e.g., S1 nuclease, or RNase (e.g., RNase A) to remove ribonucleotides from fragments. Alternatively, the double stranded products can be blunt ended by the use of a polymerase that comprises single stranded exonuclease activity such as for example T4 DNA polymerase, or any other polymerase comprising single stranded exonuclease activity or a combination thereof to degrade the overhanging single stranded ends of the double stranded products. The polymerase comprising single stranded exonuclease activity can be incubated in a reaction mixture that does or does not comprise one or more dNTPs. A combination of single stranded nucleic acid specific exonucleases and one or more polymerases can be used to blunt end the double stranded fragments generated by fragmenting the sample comprising nucleic acids. The nucleic acid fragments can be made blunt ended by filling in the overhanging single stranded ends of the double stranded fragments. For example, the fragments may be incubated with a polymerase such as T4 DNA polymerase or Klenow polymerase or a combination thereof in the presence of one or more dNTPs to fill in the single stranded portions of the double stranded fragments. The double stranded DNA fragments can be made blunt by a combination of a single stranded overhang degradation reaction using exonucleases and/or polymerases, and a fill-in reaction using one or more polymerases in the presence of one or more dNTPs.

In some cases, the 5′ and/or 3′ end nucleotide sequences of fragmented nucleic acids are not modified or end-repaired prior to ligation with the adapter oligonucleotides. For example, fragmentation by a restriction endonuclease can be used to leave a predictable overhang. In some instances, ligation with one or more adapter oligonucleotides comprising an overhang complementary to the predictable overhang on a nucleic acid fragment can be performed after fragmentation. In another example, cleavage by an enzyme that leaves a predictable blunt end can be followed by ligation of blunt-ended nucleic acid fragments to adapter oligonucleotides comprising a blunt end. End repair can be followed by an addition of at least 1, 2, 3, 4, 5, 6, 7, 8, 9, 10, 11, 12, 13, 14, 15, 16, 17, 18, 19, or 20 nucleotides, such as one or more adenine, one or more thymine, one or more guanine, one or more cytosine, or one or more of a nuclease with an artificial structure as described herein to produce an overhang. Nucleic acid fragments having an overhang can be joined to one or more adapter oligonucleotides having a complementary overhang, such as in a ligation reaction. As a non-limiting example, a single adenine can be added to the 3′ ends of end repaired DNA fragments using a template independent polymerase, followed by ligation to one or more adapters each having a thymine at a 3′ end. Adapter oligonucleotides can be joined to blunt end double-stranded nucleic acid fragments which have been modified by extension of the 3′ end with one or more nucleotides followed by 5′ phosphorylation. Extension of the 3′ end can be performed with a polymerase such as for example Klenow polymerase or any of the suitable polymerases provided herein, or by use of a terminal deoxynucleotide transferase, in the presence of one or more dNTPs in a suitable buffer containing magnesium. Nucleic acid fragments having blunt ends can be joined to one or more adapters comprising a blunt end. Phosphorylation of 5′ ends of nucleic acid fragments can be performed for example with T4 polynucleotide kinase in a suitable buffer containing ATP and magnesium. The fragmented nucleic acid molecules may be treated to dephosphorylate 5′ ends or 3′ ends, for example, by using enzymes such as phosphatases.

In some cases, a sample can be treated with an enzyme that can remove a damaged base. Enzymes involved in base excision repair can be amenable to remove damaged bases. In some instances, the enzyme can be a DNA glycosylases. DNA glycosylases can flip the damaged base out of the double helix, and cleave the N-glycosidic bond of the damaged base, leaving an apurinic/apyrimidinic (AP) site. Examples of DNA glycosylases can include Ogg1, Mag1, and Uracil-N-glycosylase. In some instances, an AP endonucleases can cleave an AP site to yield a 3′ hydroxyl adjacent to a 5′ deoxyribosephosphate. Examples of AP endonucleases can include Apn1, Apn2, APEX1 and APEX2. In some instances, a DNA polymerase as described herein can be added to repair the nucleic acid after base excision.

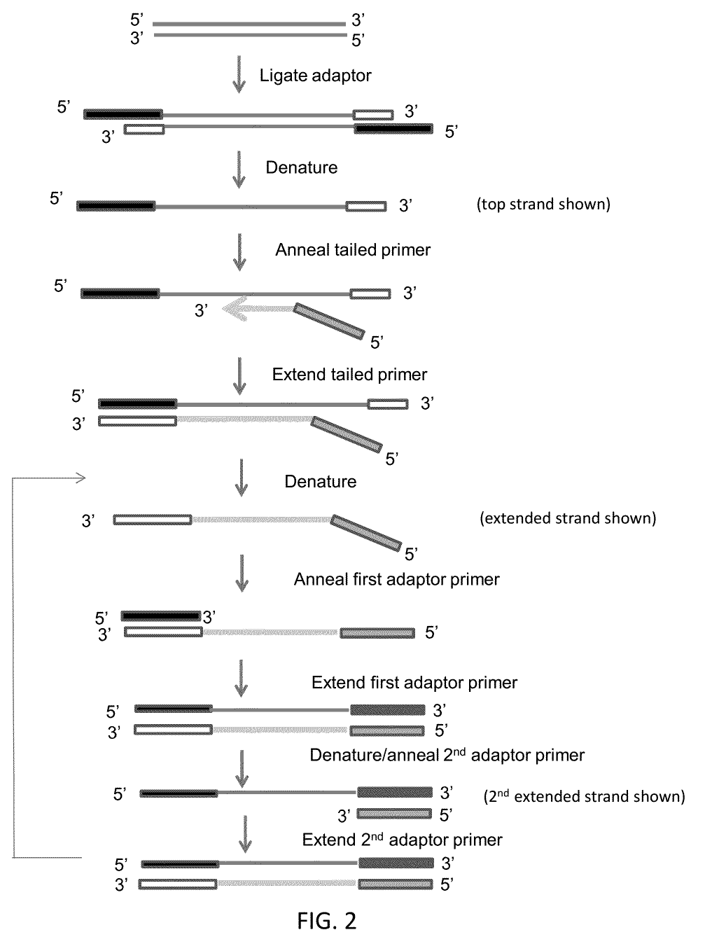

The fragmented polynucleotides can be subjected to adaptor ligation. The fragmented polynucleotides can be subjected to adaptor ligation using methods described in US20130231253, which is hereby incorporated by reference. For example, the fragmented polynucleotides may be ligated to a first adaptor sequence. The first adaptor sequence may be ligated a 5′ end or both the 5′ and 3′ ends of the polynucleotide fragments. For example, the fragmented polynucleotides can be adaptor-ligated with a forward adaptor on both 5′ and 3′ ends of the fragments (see, e.g., FIG. 2).

The amount of nucleic acid in a sample can at most, or at least, 1 pg, 10 pg, 100 pg, 1 ng, 10 ng, 100 ng, 1 ug, 10 ug, 100 ug. The amount of nucleic acid in a sample can be about 1 pg to about 10 pg, about 10 pg to about 100 pg, about 100 pg to about 1 ng, about 1 ng to about 10 ng, about 10 ng to about 100 ng, about 100 ng to about 1 ug, about 1 ug to about 10 ug, or about 10 ug to about 100 ug.

Exemplary Primers

A plurality of primers as described herein can comprise sub-pluralities of primers. Each sub-plurality of primers can be designed to target sequences corresponding to genomic regions suspected of harboring a particular class of genomic alterations. Exemplary classes of genomic alterations include, but are not limited to of single nucleotide polymorphisms (SNPs), insertions, deletions, alternative splicing events, gene fusion events, altered expression levels, copy number variations, copy number alterations, inversions, and translocations.

In some embodiments, a primer can be designed to anneal to a target at a given melting temperature (Tₘ). In some instances, a Tₘ can be from about 20 to about 100° C., about 20 to about 90° C., about 20 to about 80° C., about 20 to about 70° C., about 20 to about 60° C., about 20 to about 50° C., about 20 to about 40° C., or about 20 to about 30° C. In some instances, a Tₘ can be at least, at most, or about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 83, 84, 85, 96, 97, 98, 99 or 100° C. A plurality of primers, or primers within a sub-plurality of a plurality of primers, can be designed to have Tₘs within a range, e.g., within a range spanning 15° C., 10° C., 9° C., 8° C., 7° C., 6° C., 5° C., 4° C., 3° C., 2° C., or 1° C. A plurality of primers, or primers within a sub-plurality of a plurality of primers, can be designed to have identical Tₘs.

In some embodiments, a primer can be designed to be a certain length. In some instances, a primer can be from about 8 to about 100, from about 8 to about 90, from about 8 to about 80, from about 8 to about 70, from about 8 to about 60, from about 8 to about 50, from about 8 to about 40, from about 8 to about 30, from about 8 to about 20, or from about 8 to about 10 bases in length. In some instances, a primer can be from about 25 to about 80, from about 25 to about 75, from about 25 to about 70, from about 25 to about 65, from about 25 to about 60, from about 25 to about 55, from about 25 to about 50, from about 25 to about 45, from about 25 to about 40, from about 25 to about 35, or from about 25 to about 30 bases in length. In some instances, a primer can be at least 20, at least 25, at least 30, at least 35, at least 40, at least 45, at least 50, at least 55, at least 60, at least 65, at least 70, at least 75, at least 80, at least 85, at least 90, at least 95 or at least 100 bases in length.

In some cases, primers within a sub-plurality are not designed to have a common sequence, e.g., a barcode, that can be used to parse or bin sequence reads based on genomic locations suspected of harboring a first class of genomic alterations. In some cases, a plurality of primers comprise a barcode capable of distinguishing primers used with different samples, and have, or lack, a barcode designed to parse or bin sequence reads based on genomic locations suspected of harboring a first class of genomic alterations.

In some cases, primers within a sub-plurality are designed to have a common sequence, e.g., a barcode that can be used to parse or bin sequence reads based on genomic locations suspected of harboring a first class of genomic alterations. The barcode can be about 2, 3, 4, 5, 6, 7, 8, 9, 10, 11, 12, 13, 14, 15 nt long. The barcode can be less than 3, 4, 5, 6, 7, 8, 9, 10, 11, 12, 13, 14, 15 nt long. The barcode can use a combination of all four canonical bases (ACGT), a combination of three bases of ACGT, or a combination of two bases of ACGT. Different sub-pluralities of primers can have different length barcodes. The barcode, or the complement of the barcode, can be incorporated into an amplification product, e.g., an amplification product of a target enriched library, and the barcode, or complement of the barcode, can become part of a sequence read. Sequence reads can be partitioned or binned based on having a common barcode (or complement of the barcode).

A plurality of primers can comprise any number of sub-pluralities of primers. For example, a plurality of primers can include at least, at most, or about 2, 3, 4, 5, 6, 7, 8, 9, 10, 11, 12, 13, 14, 15, 16, 17, 18, 19, 20, 21, 22, 23, 24, 25, 26, 27, 28, 29, 20, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 sub-pluralities of primers. A plurality of primers can include about 2 to about 8, about 5 to about 20, about 10 to about 50, about 30 to about 100, or more than 100 sub-pluralities of primers. Any sub-plurality of primers can comprise any number of primers. For example, a sub-plurality of primers can include at least, at most, or about 2, 3, 4, 5, 6, 7, 8, 9, 10, 11, 12, 13, 14, 15, 16, 17, 18, 19, 20, 21, 22, 23, 24, 25, 26, 27, 28, 29, 20, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, 600, 700, 800, 900, 1000, 1100, 1200, 1300, 1400, 1500, 1600, 1700, 1800, 1900, 2000, 3000, 4000, 5000, 6000, 7000, 8000, 9000, 10000, 11000, 12000, 13000, 14000, 15000, 16000, 17000, 18000, 19000, 20000, 25000, 30000, 35000, 40000, 45000, 50000, 55000, 60000, 65000, 70000, 75000, 80000, 85000, 90000, 95000, 100000, 150000, 200000, 250000, 300000, 350000, 400000, 450000, 500000, 550000, 600000, 650000, 700000, 750000, 800000, 850000, 900000, 950000, 1000000, 1500000, 2000000, 2500000, 3000000, 3500000, 4000000, 4500000, 5000000, 5500000, 6000000, 6500000, 7000000, 7500000, 8000000, 8500000, 9000000, 9500000, or 10000000 primers. A subplurality of primers can include about 10 to about 100, about 100 to about 1000, about 1000 to about 10,000, about 10,000 to about 100,000, about 100,000 to about 1,000,000, or about 1,000,000 to about 10,000,000 different primers.

In some embodiments, a primer can anneal to a region of a target sequence corresponding to a genomic region comprising a lesion. A primer can anneal to a region of a target sequence corresponding to a genomic region that does not comprise a lesion. The term “lesion” as used herein can comprise any of the genomic alterations described herein. In some instances, a primer can anneal to a target sequence 5′ or 3′ to a lesion. In some instances, a 3′ end of a primer can anneal to a target sequence at a distance of at least 1, 2, 3, 4, 5 6, 7, 8, 9, 10, 11, 12, 13, 14, 15, 16, 17, 18, 19, 20, 21, 22, 23, 24, 25, 26, 27, 28, 29, 20, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900 or 2000 bases or base pairs away from a lesion. In some instances, a 3′ end of a primer can anneal to a target sequence at a distance of at most 1, 2, 3, 4, 56, 7, 8, 9, 10, 11, 12, 13, 14, 15, 16, 17, 18, 19, 20, 21, 22, 23, 24, 25, 26, 27, 28, 29, 20, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900 or 2000 bases or base pairs away from a lesion.

In some embodiments, a primer can be designed to anneal to a specific region of a target nucleic acid. In some instances, the 3′ end of a primer designed to anneal to a target sequence comprising a gene fusion event can anneal a base 3′ of an exon junction.

In some instances, the 3′ end of primer designed to anneal to a target sequence comprising a SNP can anneal immediately 3′ of the SNP.

In some instances, primers designed to anneal to a target sequence comprising a copy number alteration can tile across the sequence comprising the copy number alteration, e.g., a gene intron, exon, or both. The term “tile” can refer to annealing multiple primers along a target nucleic acid, wherein the annealed primers are separated by a distance. In some instances, tiling can comprise annealing primers to sequence in which pairs of annealed primers are separated by at least 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 500, 1000, 5000, or 10,000 bases. In some instances, tiling can comprise annealing at least 1, 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 550, 600, 650, 700, 750, 800, 850, 900, 950, 1000, 1100, 1200, 1300, 1400, 1500, 1600, 1700, 1800, 1900, 2000, 2500, 3000, 3500, 4000, 4500, 5000, 5500, 6000, 6500, 7000, 7500, 8000, 8500, 9000, 9500 or 10000 primers to a sequence.

In some embodiments, a primer can anneal to an exon of a target sequence corresponding to a genomic region suspected of harboring a particular class of genomic alterations. In some embodiments, a primer can anneal to an intron that is 5′ or 3′ of a target sequence corresponding to a genomic region suspected of harboring a particular class of genomic alterations. In some embodiments, a primer can anneal to an intron that is 5′ or 3′ of a target sequence corresponding to a genomic region suspected of harboring a particular class of genomic alterations. In some embodiments, a primer can anneal to a promotor region of a target sequence corresponding to a genomic region suspected of harboring a particular class of genomic alterations. In some embodiments, a primer can anneal to an enhancer region of a target sequence corresponding to a genomic region suspected of harboring a particular class of genomic alterations. In some embodiments, a primer can anneal to an operon. In some embodiments, the operon can be a lac operon. In some embodiments, a primer can anneal to an untranslated region of an RNA template. In some embodiments, a primer can anneal to an untranscribed region of a DNA template.

Primers in any one of the sub-pluralities can comprise a target-selective sequence. Such primers can also comprise an adaptor sequence. The adaptor sequence can be distinct from the adaptor sequence ligated to sample polynucleotides described herein. For example, if sample polynucleotides are ligated to a forward sequencing adaptor, the primers can comprise a reverse sequencing adaptor. Likewise, if sample polynucleotides are ligated to a reverse sequencing adaptor, the primers can comprise a forward sequencing adaptor. The primers can comprise an adaptor sequence at a 5′ end and a target-selective sequence at a 3′ end. Primers in any given sub-plurality can further comprise an adaptor or index sequence which identifies the sub-plurality.

In some instances, a primer can comprise a 5′ tail. A 5′ tail can comprise a nucleotide sequence that is different than a target sequence or that does not anneal to a target sequence. A 5′ tail sequence can be unhybridized to other sequence, e.g., other adaptor sequence or to another nucleic acid strand. In some embodiments, a 5′ tail can comprise a second adapter sequence. In some instances, a first adapter sequence and a second adapter sequence can be the same sequence. In other instances, a first adapter sequence and a second adapter sequence can be different sequences. In some embodiments, a 5′ tail can comprise an indexing adapter as described in U.S. Pat. No. 8,053,192, the contents of which are incorporated by reference herein. In some embodiments, a 5′ tail can be attached to a solid support. Non-limiting examples of solid supports that can be employed are described in U.S. Pat. No. 6,913,884, the contents of which are incorporated by reference herein.

Primers (or probes) can be synthesized using chemical synthesis, e.g., using phosphoramidite method.

Target Enrichment

Primers, for example, different pools of sub-plurality of primers, described herein can be pooled in a reaction mixture for target enrichment. The reaction mixture can comprise components for performing primer annealing and extension of target genomic regions. The reaction can produce extension products. The extension products can be subjected to an amplification reaction. The amplification reaction can be exponential, and can be carried out at various temperature cycles or isothermal. The amplification can be polymerase chain reaction. The amplification reaction can be isothermal. The oligonucleotide extension product can comprise first adaptor sequence on one end and second adaptor sequence on the other end as generated by the methods described herein. The oligonucleotide extension product can be separated from the template nucleic acid fragment in order to generate a single stranded oligonucleotide extension product with first adaptor sequence on the 5′ end and second adaptor sequence on the 3′ end. The single stranded oligonucleotide extension product can then be amplified using a first primer comprising sequence identical to the first adaptor and a second primer comprising sequence complementary to the second adaptor sequence. In this manner only oligonucleotide extension products comprising both the first and the second adaptor sequence will be amplified and thus enriched. The first adaptor and/or the second adaptor sequence can comprise an identifier sequence. The identifier sequence can be barcode sequence. The barcode sequence can be the same or different for the first adaptor and the second adaptor sequence. The first adaptor and/or the second adaptor sequence can comprise sequence that can be used for downstream applications such as, for example, but not limited to, sequencing. The first adaptor and/or the second adaptor sequence can comprise flow cell sequences which can be used for sequencing with the sequencing method developed by Illumina and described herein.

FIG. 2 depicts an exemplary embodiment of a method described herein for targeted enrichment of a polynucleotide sample. A single forward adaptor (e.g., a double stranded adaptor or single stranded adaptor) can be ligated to both ends of polynucleotide fragments (e.g., a double-stranded DNA fragment or single stranded DNA fragment) in the sample to produce forward adaptor-ligated fragments. The single forward adaptor can be a common adaptor. The forward adaptor-ligated fragments can be subjected to an end repair reaction to produce blunt ends. The forward adaptor-ligated fragments can then be denatured to generate a denatured library comprising single-stranded forward adaptor-ligated fragments with complementary ends. Custom target-selective primers, e.g., as described herein, e.g., with a reverse adaptor tail, can then be annealed to the single-stranded forward adaptor-ligated fragments comprising target genomic regions suspected of harboring specific classes of genomic alterations. Custom target-selective primers can comprise reverse adaptor tails at a 5′ portion of the primer and target-selective sequences at a 3′ portion of the primer. The reverse adaptor can comprise a distinct sequence from the forward adaptor. After annealing, the custom primers can be extended, thereby producing extension products comprising a forward adaptor sequence at a first end and a reverse adaptor sequence at a second end. The extension products can then be selectively amplified using a forward primer which anneals to the forward adaptor and a reverse primer which anneals to the reverse adaptor. Polynucleotides which were not extended with the custom primers will generally not be amplified using such primers.

Other target enrichment methods are described in U.S. patent application Ser. No. 14/836,936, which is incorporated by reference in its entirety.

A target enrichment technique employed in methods or systems provided herein can be an Agilent SureSelect Target Enrichment System. The Agilent SureSelect target enrichment system can involve use of an RNA probe set, complementary to target regions. The probe set can be designed using and online tool. Sheared DNA can be hybridized to RNA probes. Different pools of RNA probes can be designed based on different classes of genomic alterations. The RNA probes can be 120-mer biotinylated cRNA baits. Captured fragments can be separated, e.g., using streptavidin-coated beads, e.g., streptavidin coated magnetic beads. Beads can be washed, and RNA can be digested. Adaptors can be added to captured fragments, and selected regions can be amplified, e.g., by PCR. A resulting library can be quantified, e.g., using an Agilent 2100 Bioanalyzer. The library can be sequenced, e.g., using a sequencing technique described herein. Sequence reads can be parsed based on the different classes of probes used.

A target enrichment technique employed in methods or systems provided herein can be a NimbleGen SeqCap EZ choice system. A nucleic acid probe set, e.g., a DNA probe set, complementary to target regions can be designed. Different pools of probes (e.g., DNA probes) can be designed based on different classes of genomic alterations. The probes, e.g., DNA probes, can be biotinylated. An in-solution target enrichment can be performed. A nucleic acid sample, e.g., a genomic DNA sample, can be fragmented. Adaptors can be ligated to the fragments, e.g., genomic DNA fragments. The biotinylated probes can be hybridized to the fragments ligated to adaptors. The hybridized fragments can be captured, e.g., on a bead, washed, and amplified. The resulting library can be quantified, e.g., using an Agilent 2100 Bioanalyzer. The library can be sequenced, e.g., using a sequencing technique described herein. Sequence reads can be parsed based on the different classes of probes used.

For a hybridization based assay, coded adaptors can be paired with probes for specific classes of genomic alterations. For example, a barcoded Adaptor A can be attached to fragments, and a sub-plurality of primers can be used to primer reactions for target sequences suspected of comprising a first class of genomic alterations (Reaction A). In a separate reaction, barcoded Adaptor B can be attached to fragments and a sub-plurality of primers can be used to prime reactions for target sequences suspected of comprising a second class of genomic alterations (Reaction B). Products from Reaction A and Reaction B can be pooled, sequence reads can be generated, and the sequence reads can be parsed or binned based on barcodes in Adaptor A and Adaptor B.

In some embodiments, a target sequence can be enriched through conjugation to an affinity tag. In some instances, a target sequence can be coupled to biotin, which can then be purified by contacting the target sequence with streptavidin resin. Conjugation of polypeptide purification tags can also be used to enrich the target sequence. Examples of polypeptide purification tags can include an HA tag, a 6×Histidine tag, a maltose binding protein (MBP) tag or a thrioredoxin reductase (Trx) tag.

In some embodiments, a target sequence can comprise an epitope recognized by a DNA binding protein, which can then be enriched by contacting the nucleic acid with the DNA binding protein, where the DNA binding protein can be immobilized in a purification column for example. Alternatively, DNA bound to a DNA binding protein can be immunoprecipitated and separated from the bulk population, thereby enriching the population of target nucleic acids that are bound to the immunoprecipitated protein.

In some embodiments, a target nucleic acid can be enriched by washing a solution of DNA molecules over an array of specific immobilized primers designed to capture and enrich target sequences. After washing, each captured target nucleic acid can be amplified using a common primer, thereby enriching a desired population based on complementarity to the array.

Another method for enrichment of target sequences is through the incorporation of thionucleotides (see U.S. Pat. No. 5,525,471). Deoxynucleusides analogs comprising thionucleotides can be incorporated into a target sequence subjected to an amplification reaction. The selective incorporation of these thionucleotides can provide resistance to an exonuclease, while sequences lacking the thionucleotides can be digested by the endonuclease, thereby enriching a target nucleic acid sequence.

In some embodiments, a target sequence can be enriched through selective PCR amplification. A primer can be designed to confer an adaptor at a 3′ end of a target sequence upon annealing to and extending from a target sequence. A target specific primer can then be added that can be designed to anneal to a specific target, which is then enriched exponentially through successive rounds of PCR.

In some embodiments, amplification of a target sequence can comprise at least part of a genome of an organism. In some embodiments, at least 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 90%, 95%, 96%, 97%, 98%, 99% or 100% of the genome of an organism can be enriched (e.g., amplified) and analyzed.

In some embodiments, enrichment (e.g., amplification) of a target sequence can comprise enriching at least part of a transcriptome of an organism. In some embodiments, at least 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 90%, 95%, 96%, 97%, 98%, 99% or 100% of a transcriptome of an organism can be amplified and analyzed.

Sequencing

The target-enriched sample can be subjected to sequencing. Sequencing can utilize any sequencing method, such as next-generation sequencing. For example, sequencing can utilize the method commercialized by Illumina, as described U.S. Pat. Nos. 5,750,341; 6,306,597; and 5,969,119, which are hereby incorporated by reference. In general, double stranded fragment polynucleotides can be prepared by the methods described herein to produce amplified nucleic acid sequences tagged at one (e.g., (A)/(A′) or both ends (e.g., (A)/(A′) and (C)/(C′)). Single stranded nucleic acid tagged at one or both ends can be amplified (e.g., by SPIA or linear PCR).The resulting nucleic acid can then be denatured and the single-stranded amplified polynucleotides can be randomly attached to the inside surface of flow-cell channels. Unlabeled nucleotides can be added to initiate solid-phase bridge amplification to produce dense clusters of double-stranded DNA. To initiate the first base sequencing cycle, four labeled reversible terminators, primers, and DNA polymerase can be added. After laser excitation, fluorescence from each cluster on the flow cell can be imaged. The identity of the first base for each cluster can then be recorded. Cycles of sequencing can be performed to determine the fragment sequence one base at a time.

Sequencing can comprise use of sequencing by ligation methods commercialized by Applied Biosystems (e.g., SOLiD sequencing). Methods provided herein can be useful for preparing target polynucleotides for sequencing by synthesis using the methods commercialized by 454/Roche Life Sciences, including but not limited to the methods and apparatus described in Margulies et al., Nature (2005) 437:376-380 (2005); and U.S. Pat. Nos. 7,244,559; 7,335,762; 7,211,390; 7,244,567; 7,264,929; and 7,323,305. Methods provided herein can be useful for preparing target polynucleotide(s) for sequencing by the methods commercialized by Helicos BioSciences Corporation (Cambridge, Mass.) as described in, e.g., U.S. application Ser. No. 11/167,046, and U.S. Pat. Nos. 7,501,245; 7,491,498; 7,276,720; and in U.S. Patent Application Publication Nos. US20090061439; US20080087826; US20060286566; US20060024711; US20060024678; US20080213770; and US20080103058.

Methods provided herein can be useful for preparing target polynucleotide(s) for sequencing by the methods commercialized by Pacific Biosciences as described in, e.g., U.S. Pat. Nos. 7,462,452; 7,476,504; 7,405,281; 7,170,050; 7,462,468; 7,476,503; 7,315,019; 7,302,146; 7,313,308; and US Application Publication Nos. US20090029385; US20090068655; US20090024331; and US20080206764. Each of four DNA bases can be attached to one of four different fluorescent dyes. These dyes can be phospholinked. A single DNA polymerase can be immobilized with a single molecule of template single stranded DNA at the bottom of a zero-mode waveguide (ZMW). A ZMW can be a confinement structure which enables observation of incorporation of a single nucleotide by DNA polymerase against the background of fluorescent nucleotides that can rapidly diffuse in an out of the ZMW (in microseconds). It can take several milliseconds to incorporate a nucleotide into a growing strand. During this time, the fluorescent label can be excited and produce a fluorescent signal, and the fluorescent tag can be cleaved off. The ZMW can be illuminated from below. Attenuated light from an excitation beam can penetrate the lower 20-30 nm of each ZMW. A microscope with a detection limit of 20 zeptoliters (10^{˜21} liters) can be created. The tiny detection volume can provide 1000-fold improvement in the reduction of background noise. Detection of the corresponding fluorescence of the dye can indicate which base was incorporated. The process can be repeated.

Sequencing can comprise use of nanopore sequencing (see e.g. Soni G V and Meller A. (2007) Clin Chem 53: 1996-2001). A nanopore can be a small hole of the order of 1 nanometer in diameter. Immersion of a nanopore in a conducting fluid and application of a potential across it can result in a slight electrical current due to conduction of ions through the nanopore. The amount of current that flows can be sensitive to the size of the nanopore. As a DNA molecule passes through a nanopore, each nucleotide on the DNA molecule obstructs the nanopore to a different degree. Thus, the change in the current passing through the nanopore as the DNA molecule passes through the nanopore can represent a reading of the DNA sequence.

Sequencing can comprise use of semiconductor sequencing, e.g., as provided by Ion Torrent (e.g., using the Ion Personal Genome Machine (PGM)). Ion Torrent technology can use a semiconductor chip with multiple layers, e.g., a layer with micro-machined wells, an ion-sensitive layer, and an ion sensor layer. Nucleic acids can be introduced into the wells, e.g., a clonal population of single nucleic can be attached to a single bead, and the bead can be introduced into a well. To initiate sequencing of the nucleic acids on the beads, one type of deoxyribonucleotide (e.g., dATP, dCTP, dGTP, or dTTP) can be introduced into the wells. When one or more nucleotides are incorporated by DNA polymerase, protons (hydrogen ions) can be released in the well, which can be detected by the ion sensor. The semiconductor chip can then be washed and the process can be repeated with a different deoxyribonucleotide. A plurality of nucleic acids can be sequenced in the wells of a semiconductor chip. The semiconductor chip can comprise chemical-sensitive field effect transistor (chemFET) arrays to sequence DNA (for example, as described in U.S. Patent Application Publication No. 20090026082). Incorporation of one or more triphosphates into a new nucleic acid strand at the 3′ end of the sequencing primer can be detected by a change in current by a chemFET. An array can have multiple chemFET sensors.

Sequencing can produce sequence reads. In some embodiments, a sequence read can have a penetrance of at least 100, 150, 200, 250, 300, 350, 400, 450, 500, 600, 700, 800, 900, 1000, 1100, 1200, 1300, 1400, 1500, 1600, 1700, 1800, 1900, 2000, 2500, or 3000 base pairs from a region where a sequencing primer anneals to a target sequence. In some embodiments, a sequence read can have a penetrance of about 100 to about 3000, about 100 to about 2500, about 100 to about 2000, about 100 to about 1900, about 100 to about 1800, about 100 to about 1700, about 100 to about 1600, about 100 to about 1500, about 100 to about 1400, about 100 to about 1300, about 100 to about 1200, about 100 to about 1100, about 100 to about 1000, about 100 to about 900, about 100 to about 800, about 100 to about 700, about 100 to about 600, about 100 to about 500, about 100 to about 450, about 100 to about 400, about 100 to about 350, about 100 to about 300, about 100 to about 250, about 100 to about 200, or about 100 to about 150.

In some embodiments, sequencing can produce at least one sequence read. In some embodiments, sequencing can produce at least 1, 2, 3, 4, 5, 6, 7, 8, 9 or 10 sequencing reads. In such embodiments, a computer processor can separate each sequence read into at least 1, 2, 3, 4, 5, 6, 7, 8, 9 or 10 data files. Each individual data file can be compiled and analyzed to determine the presence of at least 1, 2, 3, 4, 5, 6, 7, 8, 9 or 10 classes of genomic aberrations. The number of sequence reads from a sample can be about, more than, less than, or at least 100, 1000, 5,000, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, 1,000,000, 2,000,000, 3,000,000, 4,000,000, 5,000,000, 6,000,000, 7,000,000, 8,000,000, 9,000,000, or 10,000,000.

In some embodiments, a “sequence depth” can be described as the number of times a nucleotide is read in a sequencing process. In some instances, the sequencing depth of a sequencing process described herein can be at least, or about 1, 2, 3, 4, 5, 6, 7, 8, 9, 10, 50, 100, 500, 1000, 5000, or 10,000 times. In some instances, a sequence depth can be about 10× to about 100×, about 50× to about 500×, about 100× to about 1000×, or about 1000× to about 10,000×.

In some embodiments, the sequencing processes described herein can process multiple samples simultaneously. In some embodiments, a sequencing process can process at least 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 300, 400, 500, 600, 700, 800, 900, 1000, 1100, 1200, 1300, 1400, 1500, 1600, 1700, 1800, 1900 or 2000 samples simultaneously.

In some embodiments, a sequencing process can be used in conjunction with other techniques, e.g., other techniques described herein. In some embodiments, a sequencing process can be used in conjunction with a target enrichment technique as described herein (e.g. PCR, streptavidin binding, etc.), which can then be sequenced after enrichment to positively or negatively identify genomic alterations in a target nucleic acid. In some embodiments, a sequencing process can be used in conjunction with a karyotyping technique. As a non-limiting example, fluorescence in situ hybridization can be used as a karyotyping tool to determine the relative number of nucleic acid sequences in a test cell as described in U.S. Pat. No. 6,197,501. This analysis can be used to detect any of the genomic aberrations described herein, which can be coupled to a sequencing process described herein to positively identify the genomic aberration.

Sequence Read Analysis

Sequence reads generated using methods or systems described herein can be queried for presence or absence of a sequence corresponding to any one of the sub-pluralities of primers described herein. Such presence or absence can be used to identify sequence reads generated from templates resulting from extension using a particular sub-plurality of primers. Sequence reads generated from templates resulting from specific sub-pluralities of primers can then be subjected to sequence analysis for detection of specific classes of genomic alterations. The querying, transferring, and analysis can be implemented using a computer readable medium comprising computer executable code. The computer readable medium can further comprise a database of identifying sequences for each sub-plurality of primers. The database can be, for example, a file, e.g., a data file, or a list. In some cases, sequence read analysis comprises removing duplicates, e.g., PCR duplicates.

Sequence Alignment

Sequence reads can be aligned to a reference genome. A reference genome can be, e.g., Hg18, Hg19, GRch37 or GRch38. A reference genome can be from a human. A reference genome can be from a human with a condition, e.g., cancer.

Sequence reads collected by a sequencing process described herein can be aligned using any of a number of algorithms. In some embodiments, a global alignment can be used. In some specific embodiments, a global alignment can employ a Needleman-Wunsch algorithm. In some embodiments, a local alignment can be used. In some specific embodiments, a local alignment can employ a Smith-Waterman algorithm. In some embodiments, a glocal alignment, which is a hybrid local and global algorithm, can be used. In some embodiments, a pairwise alignment can be used when comparing two sequence reads at a time to each other. In some embodiments, a multiple sequence alignment can be used when comparing multiple sequences to each other.

In some instances, a dynamic programming can be used with any of the sequencing alignment algorithms described herein. In some embodiments, dynamic programming can be used to apply a variable gap penalty to a sequence alignment, and therefore may facilitate alignments in the presence of frameshift mutations. In some embodiments, a progressive method can be used to align sequences of greater similarity in a multiple sequence alignment first, followed by alignment of progressively less similar sequences. This analysis can produce “weight” into a dataset by aligning similar sequences first, which can reduce the error associated with alignment of more dissimilar sequences. In some specific embodiments, the progressive method can be a Clustal method. In some instances, iterative methods can be employed, which can be used to align a sequence initially, and subsequently realign the sequence reads iteratively, which can be used to eliminate bias in the initial alignment.

Sequence alignment software that can be used in methods or systems described herein, e.g., for database searching can include, e.g., BLAST, CS-BLAST, CUDASW++, DIAMOND, FASTA, GGSEARCH, GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE.

Sequence alignment software that can be used in methods or systems provided herein, e.g., for pairwise alignment, can include ACANA, AlignMe, Bioconductor, BioPerl dpAlign, BLASTZ, LASTZ, CUDAlign, DNADot, DOTLET, FEAST, Genome Compiler, G-PAS, GapMis, GGSEARCH, GLSEARCH, JAligner, K*Sync, LALIGN, NW-align, mAlign, matcher, MCALIGN2, MUMmer, needle, Ngila, NW, parasail, Path, PatternHunter, ProbA (propA), PyMOL, REPuter, SABERTOTTH, Satsuma, SEQALN, SIM, GAP, NAP, LAP, SIM, SPA (Super pairwise alignment), SSEARCH, Sequences Studio, SWIFT suit, stretcher, tranalign, UGEN, water, wordmatch or YASS.

Sequence alignment software that can be used in methods or systems provided herein, e.g, for multiple sequence alignment, can include ABA, ALE, AMAP, anon, BAli-Phy, Base-By-Base, BHAOS/DIALIGN, Bowtie, Bowtie 2, BWA, ClustalW, CodonCode Aligner, Comass, DECIPHER, DIALIGN-TX, DIALIGN-T, DNA Alignment, DNA Baser Sequence Assembler, EDNA, FSA, Geneious, Kalign, MAFFT, MARNA, MAVID, MSA, MSAProbs, MULTALIN, Multi-LAGEN, MUSCLE, Opal, Pecan, Phylo, Praline, PicXAA, POA, Probalign, ProbCons, PROMALS3D, PRRN/PRRP, PSAlign, RevTrans, SAGA, SAM, Se-Al, STAR, STAR-Fusion, StatAlign, Stemloc, T-Coffee, UGENE, VectorFriends, or GLProbs.

Sequence alignment software that can be sued in methods or systems provided herein, e.g., for genomics analysis, can include ACT (Artemis Comparison Tool), AVID, BLAT, DECIPHER, FLAK, GMAP, Splign, Mauve, MGA, Mulan, Multiz, PLAST-ncRNA, Sequerome, Sequilab, Shuffle-LAGAN, SIBsim4/Sim4, or SLAM.

Sequence alignment software that can be used in methods or systems provided herein, e.g., for short-read sequence alignment, can include BarraCUDA, BBMap, BFAST, BigBWA, BLASTN, BLAT, Bowtie, HIVE-hexagon, BWA, BWA-PSSM, CASHX, Cloudburst, CUDA-EC, CUSHAW, CUSHAW2, CUSHAW2-GPU, CUSHAW3, drFAST, ELAND, ERNE, GASSST, GeM, Genalice MAP, Geneious Assembler, GensearchNGS, GMAP, GSNAP, GNUMAP, iSSAC, LAST, MAQ, mrFAST, mrsFAST, MOM, MOSAIK, MPscan, Novoalign, NovoalignCS, NextGENe, NextGenMap, Omixon, PALMapper, Partek, PASS, PerM, PRIMEX, QPalma, RazerS, REAL, cREAL, RMAP, rNA, RTG Investigator, Segemehl, SeqMap, Shrec, SHRiMP, SLIDER, SOAP, SOAP2, SOAP3, SOAP-dp, SOCS, SSAHA, SSAHA2, Stampy, SToRM, Subread, Subjunc, Taipan, UGENE, VelociMapper, XpressAlign, or ZOOM.

Alignment viewers/editors that can be used with methods or systems described herein can include, e.g, Ale, AliView, Base-By-Base, BioEdit, BioNumerics, BoxShade, CINEMA, CLC viewer, ClustalX viewer, Cylindrical BLAST Viewer, DECIPHER, Discovery Studio, DnaSP, emacs-biomode, FLAK, Genedoc, Geneious, Integrated Genome Browser (IGB), IVistMSA, Jalview 2, JEvTrace, JSAV, Maestro, MEGA, Multiseq (vmd plugin), MView, PFAAT, Ralee, S2S RNA editor, Seaview, Sequilab, SeqPop, Sequlator, SnipViz, Strap, Tablet, UGENE, VISSA sequence/structure viewer, DNApy, or Alignment Annotator.

Open-source bioinformatics software that can be used with methods or systems described herein can include, e.g., NET Bio, AMPHORA, Anduril, Autodock, Bedtools, Biochemical Algorithms Library (BALL), Bioclipse, Bioconductor, BioJava, BioJS, BioMOBY, BioPerl, BioPHP, Biophython, BioRuby, EMBOSS, FACS, Galaxy, GenePattern, GeWorkbench, GMOD, GenGIS, GenomeSpace, GENtle, IGV, Integrated Genome Browser, InterMine, LabKey Server, LARVA, mothur, PathVisio, PromKappa, ProSSA, PyPDB, RaFoSA, Orange, Staden Package, STAMP, Taverna workbench, TRAL, UGEN, or Unipept.

Binning Sequence Reads Based on Probe Sequence

Sequences reads can be binned based on probes (primers) used to amplify target sequences. In some cases, at least, or about 2, 3, 4, 5, 6, 7, 8, 9, 10, 20, 25, 30, 50, 75, or 100 probe pools are used. In some cases, about 2 to about 10, about 2 to about 5, about 3 to about 10, or about 3 to about 6 probe pools are used. Each probe pool can be designed to detect a different class of genomic alteration. In some embodiments, a single binning method can be used to bin a library of sequence reads. In some embodiments, a combination of binning methods can be used sequentially or simultaneously to bin a library of sequence reads.

In some embodiments, a direct lookup can be used to bin sequence reads. In such an embodiment, a sequence present in a primer can be used to bin a sequence read. The known sequence of a primer can be used to query sequence reads that contain the primer sequence, or a complement of the primer sequence. In some instances, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, at least 60, at least 65, at least 70, at least 75, at least 80, at least 85, at least 90, at least 95 or at least 100 bases of a primer sequence can be used to query sequence reads. In some cases, at least 5%, 10%, 15%, 20%, 25%, 30%, 35%, 40%, 45%, 50%, 55%, 60%, 65%, 70%, 75%, 80%, 85%, 90%, 95% or 100% of a primer sequence can be used as an identifier to bin a sequence read. In some instances at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, at least 60, at least 65, at least 70, at least 75, at least 80, at least 85, at least 90, at least 95 or at least 100 bases of a primer sequence can be used as an identifier to bin a sequence read.

In some instances, a reverse sequence read can be queried for the presence of a primer sequence. In some instances, the querying of an incorporated primer sequence can be performed using automated software. In some instances, a threshold for a match during querying can allow for at least 1, at least 2, at least 3, at least 4, at least 5, at least 6, at least 7, at least 8, at least 9, or at least 10 mismatches from a known primer sequence. A threshold for binning a sequence read can be at least 50%, 60%, 70%, 75%, 80%, 85%, 90%, 91%, 92%, 93%, 94%, 95%, 96%, 97%, 98%, 99%, or 100% identity of a sequence in a sequence read and a primer sequence, or complement of a primer sequence.

In some embodiments, a Bowtie alignment can be used to bin sequence reads. In such an embodiment, an alignment index of probe sequences can be constructed. This alignment index can be used to align a library of sequence reads (e.g., reverse sequence reads) to a probe. In some instances, a sequence read can align to a probe comprising a sequence at least 10%, 15%, 20%, 25%, 30%, 35%, 40%, 45%, 50%, 55%, 60%, 65%, 70%, 75%, 80%, 85%, 90%, 95% or 100% identical to a primer sequence (or complement of a primer sequence) incorporated into the target sequence read. Each sequence aligned to a particular probe can then be binned simultaneously or sequentially based on the probe they are aligned to.

In some instances, a K-mer based method can be used to bin sequence reads. In some instances, probe sequences can be broken up into fragments (which can be called k-mers), which can be aligned against a sequence read (e.g., a reverse sequence read). In some instances, a sequence read can be broken up into fragments (k-mers), which can be aligned against a probe sequence. In some cases, a fragment (k-mer) can comprise at least 1, 2, 3, 4, 5, 6, 7, 8, 9, 10, 11, 12, 13, 14, 15, 16, 17, 18, 19 or 20 bases. A k-mer can be 8 bases. In some cases, a fragment (k-mer) can comprise at most the length of a probe (primer) or a sequence read. The number of k-mers that match a target sequence can be calculated, which can be used to bin a sequence read based on the sequence of the original, unfragmented probe. A sequence read can match a probe when it contains the most k-mer (subsequence) matches to that probe.

In some embodiments, binning can be carried out with the assistance of computer hardware. In some instances, a computer hardware can be a physical hardware. In some instances, a computer hardware can be a virtual hardware. In some cases, a computer hardware can comprise a computer processor. In some instances, the computer processor can be an INTEL® processor. An INTEL® processor can have at least 1, 2, 4 or 8 processor cores. In some instances, a computer hardware can comprise RAM memory. A computer hardware can comprise at least 4, 8, 16, 32, 64, 128, 256, 512, or 1025 GB of RAM memory. In some instances, a computer hardware can run a real-time operating system. In some instances, a computer hardware can run a single user, single task operating system. In some instances, a computer hardware can run a single user, multi-tasking operating system. In some cases, a single user, multi-tasking operating system can be a Microsoft Windows, Mac OS, or Linux based operating system. In some instances, a computer hardware can run a multi user operating system. In some cases, a multi user operating system can be a Unix operating system.

Analyzing Sequence Reads

In some instances, the number of sequence reads that can align to a target sequence can be normalized. In some instances, a normalization method can be a calculation of reads per kilobase per million (RPKM). An RPKM can be calculated by dividing the number of sequences that align to a probe by the product of the total number of sequence reads and the number of kilobases of transcript, and multiplying by 1,000,000. In some instances, a normalization method can be a calculation of fragments per kilobase of transcript per million mapped reads (FPKM). An FPKM can be calculated as described for an RPKM normalization; an FPKM can describe RNA transcripts specifically whereas an RPKM can describe nucleic acids in general. In some instances, a normalization method can be a calculation of transcripts per kilobase million (TPM). A TPM can be calculated by first dividing the number of sequence reads aligned to a target by the length of a target sequence, and dividing this by the number of the number of total sequence reads divided by 1,000,000.

In some cases, multiple algorithms described herein can be used to analyze sequence reads in a partition or bin. In some instances, at least 1, 2, 3, 4, 5, 6, 7, 8, 9, 10, 11, 12, 13, 14, 15, 16, 17, 18, 19 or 20 algorithms can be used to analyze sequence reads in a partition or bin. In some instances, at least 20, 25, 30, 35, 40, 45, 50, 55, 60, 65, 70, 75, 80, 85, 90, 95 or 100 algorithms can be used to analyze sequence reads in a partition or bin.

Sequencing reads can be separated into multiple partitions or bins. In some cases, multiple algorithms described herein can be used sequentially to analyze sequence reads in multiple partitions or bins. In some instances, multiple algorithms described herein can be used simultaneously to analyze sequence reads in multiple partitions or bins. In some instances, multiple algorithms described herein can be used sequentially to analyze sequence reads in a same partition or bin. In some instances, multiple algorithms described herein can be used simultaneously to analyze sequence reads in a same partition.

In some embodiments, multiple algorithms capable of detecting the same type of genomic alteration can be used to analyze sequence reads in the same bin; the analysis can be done sequentially or simultaneously. For example, fusions can be detected by using SOAPFuse, ChimeraScan and STAR-Fusion algorithm to analyze sequence reads in a partition or bin. In some embodiments, multiple algorithms, each capable of detecting a different type of genomic alteration, can be used simultaneously to analyze sequence reads in a partition or bin. In some instances, a fusion detection and a SNP detection algorithm can be used to analyze sequence reads in a partition or bin, sequentially or simultaneously. In some instances, the resulting data can be combined.

SNP Analysis Workflow

Exemplary SNP analysis workflows include, e.g., Bowtie 2 or BWA analysis, MuTect, SAMtools, Free Bayes, and/or Genome Analysis toolkit (GATK) best practices pipeline. Bowtie analysis can comprise implementing the Burrows-Wheeler transform for aligning. MuTect can comprise: 1. Pre-processing; 2. Statistical Analysis; 3. Post-processing. Pre-processing can comprise an initial alignment of sequencing reads. Statistical analysis can comprise using two Bayesian classifiers—the first can detect whether a SNP is non-reference at a given site and, for those sites that are found as non-reference, the second classifier can make sure the normal does not carry the SNP. Post-processing can comprise removal of artifacts of sequencing, short read alignments and hybrid capture. SAMtools can comprise storing, manipulating and aligning sequencing reads stored as SAM files. Free Bayes can comprise an alignment based on literal sequences of reads aligned to a particular target, not their precise alignment. The GATK best practices pipeline can comprise: 1. Pre-Processing; 2. Variant Discovery; and 3. Callset Refinement. Pre-Processing can comprise starting from raw sequence data, e.g., in FASTQ or uBAM format, and producing analysis-ready BAM files. Processing steps can include alignment to a reference genome as well as data cleanup operations to correct for technical biases and make the data suitable for analysis. Variant Discovery can comprise starting from analysis-ready BAM files and producing a callset in VCF format. Processing can involve identifying sites where one or more individuals display possible genomic variation, and applying filtering methods appropriate to the experimental design. Callset Refinement can comprise starting and ending with a VCF callset. Processing can involve using meta-data to assess and improve genotyping accuracy, attach additional information and evaluate the overall quality of the callset.

For SNP detection, the reverse reads corresponding to the set of reads that map to a “SNP” probe sequence file can be aligned to a genome using BWA. Variants can be detected using GATK best practices pipeline.

Analysis can be performed on a Linux system.

A SNP identified by a method described herein can be analyzed by other methods, e.g., hybridization based technique, e.g., microarray, PCR, real-time PCR, digital PCR, droplet digital PCR.

Fusion Analysis Workflow

Fusions can be detected using a fusion analysis workflow. Exemplary gene fusion analysis bioinformatics workflows include, e.g., Spliced Transcripts Alignment to a Reference (STAR) alignment and fusion detection software.

Fusion detection software can include Bellerophontes, BreakFusion, Chimera, chimerascan, chimEric TranScript detection algorithm, Complex Reads Analysis & Classification, comrad, deFuse, Dissect, FusionAnalyser, FusionCatcher, FusionFinder, FusionHunter, FusionMap, FusionMatcher, FusionQ, FusionSeq, IDP-fusion, JAFFA, NCLscan, nFuse, Pegasus, R453Plus1Toolbox, ShortFuse, STAR-Fusion, Snow-Shoes-FTD, SnowsShoes-FTD, SOAPfuse, SOAPfusion, TopHat-Fusion, Tumor-specimen suited RNA-seq Unified Pipeline, or ViralFusionSeq, or any combination thereof.

For fusion detection, forward and reverse reads corresponding to a set reads that map to a “fusion” probe sequence file can be analyzed by SOAPfuse. Fusion detection can be dependent on detecting instances where the forward and reverse reads came from different genes and where reads spanned splice junctions.

A fusion call method can comprise one or more of the following steps: 1. Trim reads of adaptor (optional); 2) assign probes to read using k-mer approach, e.g, as described herein; 3. Deduplicate raw reads using an N6 sequence in an index read (remove PCR duplicates) (optional); 4. quality trim probe-assigned reads (e.g., remove portions of reads where quality dips below a threshold) (optional); 5. align to genome with STAR; 6. filter reads which do not fully contain expected probe extension sequence (optional); 7. run STAR-Fusion with filtered and labeled STAR alignment; 8. post process called fusions based on expected contributing probes (optional).

Analysis can be performed on a Linux system.

A fusion event identified by a method described herein can be analyzed by other methods, e.g., hybridization based technique, e.g., microarray, PCR, real-time PCR, digital PCR, or droplet digital PCR.

Expression Analysis WorkFlow

Expression analysis can be carried out by two independent methods. First, forward reads corresponding to a set of reads that map to an “expression” probe sequence file can be aligned to a genome with RNA STAR. After alignment, Fragments Per Kilobase of transcript per Million mapped reads (FPKM) values for the targeted, as well as housekeeping genes, can be calculated using Cufflinks. Second, expression can be calculated using expression “probe counts,” or a number of times each “expression” probe sequence is present in a reverse read sequence. All values can be normalized between replicates and across cell lines.

DeSeq, SailFish, can also be used, or any expression analysis software for short reads can be used. In some cases, RPKMs, FPKMs, or TPMs are computed. Analysis can be performed on a Linux system. An altered expression event identified by a method described herein can be analyzed by other methods, e.g., hybridization based technique, e.g., microarray, PCR, real-time PCR, digital PCR, or droplet digital PCR.

Alternative Splicing WorkFlow

For alternative splicing, forward and reverse reads corresponding to a set of reads that map to an “exon usage” probe sequence file can be aligned to a genome with RNA STAR. Exon usage/isoform expression can be calculated using Cufflinks. Exon usage can be dependent on having forward and reverse reads map to an exon present in a gene isoform, as well as reads spanning to neighboring exons across splice junctions.

In some instances, an alignment tool capable of identifying splice sites from sequence reads can be employed. In some instances, the alignment tool can be TopHat, MapSplice, SpliceMap, HMMsplicer, GSNAP, STAR, RUM, SoapSplice or HISAT. In some instances, additional workflows can be used to predict isoform expression and exon usage. In some instances, the additional workflow can be Cuffdiff, ALEXA-seq, MISO, SplicingCompass, Flux Capacitor, JuncBASE, DEXSeq, MATS, SpliceR. FineSplice or ARH-seq. A Linux system can be used for analysis. An alternative splicing event identified by a method described herein can be analyzed by other methods, e.g., hybridization based technique, e.g., microarray, PCR, real-time PCR, digital PCR, or droplet digital PCR.

Copy Number Analysis WorkFlow

Custom counting of probe reads can be performed, e.g., after BWA or Bowtie alignment. Copy number analysis can be performed using tools such CONTRA. A Linux system can be used for analysis.

The following steps can be performed in a fusion analysis. 1. trim reads to remove adaptor and poor quality; linkers can be removed (a linker can be a sequence, e.g., from about 1 to about 20 bases, e.g., 15, that can be attached to a 5′ end of a primer (optional); 2. align the reads to the genome; 3. deduplicate reads (can be done before or after alignment) (optional); 4. count Forward reads that fall in a probePlus300 (file containing genomic coordinates for a 30 0 bp window in which coverage is expected that includes the probe and region downstream of the probe) region of each probe; 5. normalize each count by total counts overall; 6. obtain gene level copy number levels by averaging normalized counts across the gene (optional; one can also average probe counts across smaller portions of genes, ie: exons, introns); 7. obtain relative levels by comparing this number to the genes same number in a control sample (optional).

In some cases, the forward reads can be counted in each probe landing region and for each gene, the counts of all probe reads across a gene can be averaged to obtain a gene level copy number value that can be compared to a reference sample, e.g., PROMEGA™ Male (a combination of multiple individuals that can act as a two-copy reference control). Alternatively, a publically available tool, e.g., CONTRA, can be used to find significant copy number alterations.

Analysis can be performed, e.g., on a Linux system.

### Computer Systems

In another aspect, described herein are computer systems for the integrated analysis of multiple classes of genomic alterations. The computer system can provide a report communicating the analysis of the multiple classes of genomic alterations. The computer system can execute instructions contained in a computer-readable medium. The processor can be associated with one or more controllers, calculation units, and/or other units of a computer system, or implanted in firmware. One or more steps of the method can be implemented in hardware. One or more steps of the method can be implemented in software. Software routines may be stored in any computer readable memory unit such as flash memory, RAM, ROM, magnetic disk, laser disk, or other storage medium as described herein or known in the art. Software may be communicated to a computing device by any communication method including, for example, over a communication channel such as a telephone line, the internet, a wireless connection, or by a transportable medium, such as a computer readable disk, flash drive, etc. The one or more steps of the methods described herein may be implemented as various operations, tools, blocks, modules and techniques which, in turn, may be implemented in firmware, hardware, software, or any combination of firmware, hardware, and software. When implemented in hardware, some or all of the blocks, operations, techniques, etc. may be implemented in, for example, an application specific integrated circuit (ASIC), custom integrated circuit (IC), field programmable logic array (FPGA), or programmable logic array (PLA).

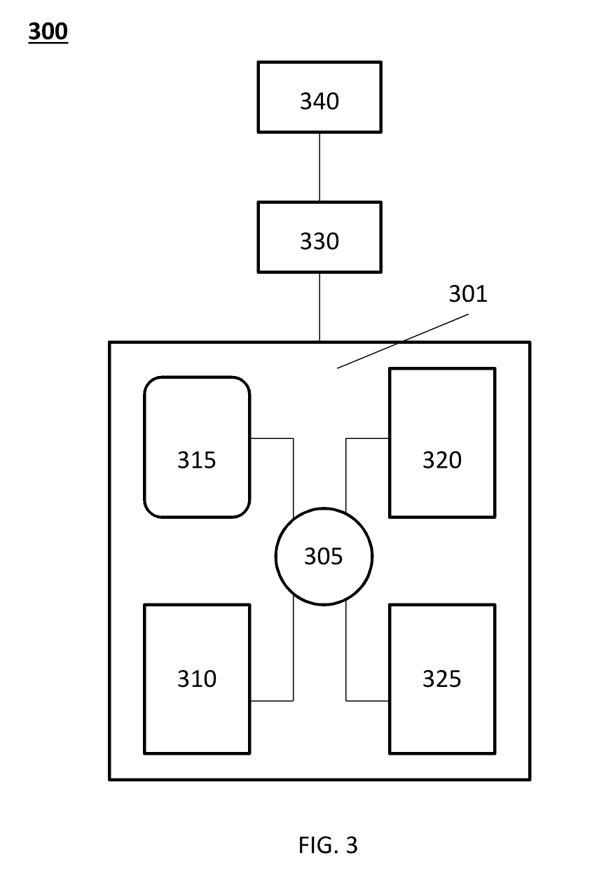

FIG. 3 depicts a computer adapted to enable a user to detect, analyze, and process sequence data. The system 300 can include a central computer server 301 that is programmed to implement exemplary methods described herein. The server 301 can include a central processing unit (CPU, also “processor”) 305 which can be a single core processor, a multi core processor, or plurality of processors for parallel processing. The server 301 also can include memory 310 (e.g. random access memory, read-only memory, flash memory); electronic storage unit 315 (e.g. hard disk); communications interface 320 (e.g. network adaptor) for communicating with one or more other systems; and peripheral devices 325 which may include cache, other memory, data storage, and/or electronic display adaptors. The memory 310, storage unit 315, interface 320, and peripheral devices 325 can be in communication with the processor 305 through a communications bus (solid lines), such as a motherboard. The storage unit 315 can be a data storage unit for storing data. The server 301 can be operatively coupled to a computer network (“network”) 330 with the aid of the communications interface 320. The network 330 can be the Internet, an intranet and/or an extranet, an intranet and/or extranet that is in communication with the Internet, a telecommunication or data network. The network 330, with the aid of the server 301, can implement a peer-to-peer network, which may enable devices coupled to the server 301 to behave as a client or a server.

The storage unit 315 can store files, such as subject reports, and/or communications with the caregiver, sequencing data, data about individuals, or any aspect of data.

The server can communicate with one or more remote computer systems through the network 330. The one or more remote computer systems may be, for example, personal computers, laptops, tablets, telephones, Smart phones, or personal digital assistants.

The system 300 can include a single server 301. In some situations, the system can include multiple servers in communication with one another through an intranet, extranet and/or the Internet.

The server 301 can be adapted to store sequence information, such as, for example, information on any of the classes of genomic alterations described herein. The server 301 can also be adapted to store, e.g., patient history and demographic data and/or other information of potential relevance. Such information can be stored on the storage unit 315 or the server 301 and such data can be transmitted through a network.

Methods as described herein can be implemented by way of machine (or computer processor) executable code (or software). The machine-executable code can be stored on an electronic storage location of the server 301, such as, for example, on the memory 310, or electronic storage unit 315. During use, the code can be executed by the processor 305. The code can be retrieved from the storage unit 315 and stored on the memory 310 for ready access by the processor 305. In some situations, the electronic storage unit 315 can be precluded, and machine-executable instructions can be stored on memory 310. Alternatively, the code can be executed on a second computer system 340.

Aspects of the systems and methods provided herein, such as the server 301, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless likes, optical links, or the like, also may be considered as media bearing the software. As used herein, unless restricted to non-transitory, tangible “storage” media, terms such as computer or machine “readable medium” can refer to any medium that participates in providing instructions to a processor for execution.

Hence, a machine readable medium, such as computer-executable code, may take many forms, including but not limited to, tangible storage medium, a carrier wave medium, or physical transmission medium. Non-volatile storage media can include, for example, optical or magnetic disks, such as any of the storage devices in any computer(s) or the like, such may be used to implement the system. Tangible transmission media can include: coaxial cables, copper wires, and fiber optics (including the wires that comprise a bus within a computer system). Carrier-wave transmission media may take the form of electric or electromagnetic signals, or acoustic or light waves such as those generated during radio frequency (RF) and infrared (IR) data communications. Common forms of computer-readable media therefore include, for example: a floppy disk, a flexible disk, hard disk, magnetic tape, any other magnetic medium, a CD-ROM, DVD, DVD-ROM, any other optical medium, punch cards, paper tame, any other physical storage medium with patterns of holes, a RAM, a ROM, a PROM and EPROM, a FLASH-EPROM, any other memory chip or cartridge, a carrier wave transporting data or instructions, cables, or links transporting such carrier wave, or any other medium from which a computer may read programming code and/or data. Many of these forms of computer readable media may be involved in carrying one or more sequences of one or more instructions to a processor for execution.

The results of the integrated analysis can be presented to a user with the aid of a user interface, such as a graphical user interface.

A computer system can be used for one or more steps, including, e.g., sample collection, sample processing, sequencing, querying sequence reads for presence of any sequence corresponding to one or more sub-pluralities of primers described herein, transferring queried sequence reads to data files according to the sub-pluralities of primers, subjecting the transferred sequence reads to specific bioinformatics workflows for analyzing a particular class of genomic alterations, receiving patient history or medical records, receiving and storing measurement data, analyzing said measurement data determine a diagnosis, prognosis, or therapeutic efficacy, generating a report, and reporting results to a receiver.

A client-server and/or relational database architecture can be used in any of the methods described herein. A client-server architecture can be a network architecture in which each computer or process on the network is either a client or a server. Server computers can be powerful computers dedicated to managing disk drives (file servers), printers (print servers), or network traffic (network servers). Client computers can include PCs (personal computers) or workstations on which users run applications, as well as example output devices as disclosed herein. Client computers can rely on server computers for resources, such as files, devices, and even processing power. The server computer can handle all of the database functionality. The client computer can have software that handles front-end data management and receive data input from users.

After performing a calculation, a processor can provide the output, such as from a calculation, back to, for example, the input device or storage unit, to another storage unit of the same or different computer system, or to an output device. Output from the processor can be displayed by a data display, e.g., a display screen (for example, a monitor or a screen on a digital device), a print-out, a data signal (for example, a packet), a graphical user interface (for example, a webpage), an alarm (for example, a flashing light or a sound), or a combination of any of the above. An output can be transmitted over a network (for example, a wireless network) to an output device. The output device can be used by a user to receive the output from the data-processing computer system. After an output has been received by a user, the user can determine a course of action, or can carry out a course of action, such as a medical treatment when the user is medical personnel. An output device can be the same device as the input device. Example output devices include, but are not limited to, a telephone, a wireless telephone, a mobile phone, a PDA, a flash memory drive, a light source, a sound generator, a fax machine, a computer, a computer monitor, a printer, an iPod, and a webpage. The user station may be in communication with a printer or a display monitor to output the information processed by the server. Such displays, output devices, and user stations can be used to provide an alert to the subject or to a caregiver thereof.

Data relating to the present disclosure can be transmitted over a network or connections for reception and/or review by a receiver. The receiver can be but is not limited to the subject to whom the report pertains; or to a caregiver thereof, e.g., a health care provider, manager, other healthcare professional, or other caretaker; a person or entity that performed and/or ordered the genotyping analysis; a genetic counselor. The receiver can also be a local or remote system for storing such reports (e.g. servers or other systems of a “cloud computing” architecture). A computer-readable medium can include a medium suitable for transmission of a result of an analysis of a biological sample.

Data storage can involve use of a ˜50TB NAS (network-attached storage). Processing can involve use multiple virtual Linux machines each with 4 dual cores (INTEL® XEON® CPU E5-26500@2) and 128 GB Ram.

Applications

In some instances, the compositions described herein can be used in a clinical setting. In some embodiments, a subject can provide a biological sample as described herein for diagnosis of a disease of known genetic genotype. In such embodiments, at least one genomic alteration can be identified by contacting a first sub-plurality of primers constructed to anneal to a target sequence corresponding to a genomic sequence suspected of harboring a first class of genomic alteration with a nucleic acid sample derived from the biological sample, followed by contacting a second sub-plurality of primers constructed to anneal to a target sequence corresponding to a genomic sequence suspected of harboring a second class of genomic alteration with the nucleic acid sample derived from the biological sample. The sequence reads can be quarried using a computer system described herein to determine the presence of the at least one genomic alteration. The data file can then be analyzed by a health care professional in order to correlate the at least one genomic alteration with a potential disease state.

In embodiments, the compositions described herein can be used to screen for specific disease states associated with a genomic alteration described herein. As a non-limiting example, a nucleic acid sample can be screened for mutations in BRCA1 or BRCA2, which may be used to diagnose a breast or ovarian cancer state. As another non-limiting example, a nucleic acid sample can be screened for mutations in p53, which may be used to diagnose various cancer states. As another non-limiting example, a nucleic acid sample can be screened for mutations in PARK2, which may be used to diagnose Parkinson disease. Other mutations in genes known to cause specific disease states can be screened in a similar manner to potentially diagnose a disease state.

In some embodiments, the disease can be of unknown genetic phenotype. In such embodiments, a genomic alteration can be identified by identified by contacting n sub-pluralities of primers engineered to anneal to n targets corresponding to potential genomic targets. In some embodiments, n can be 1, 2, 3, 4, 5, 6, 7, 8, 9, 10, 15, 20, 25, 30, 40, 50, 60, 70, 80, 90, or 100. With the aid of a computer processor described herein, each sequence read can be separated and stored in a data file, which can be interpreted by a health care professional to identify an unknown phenotype. In some embodiments, the data files can be interpreted by a researcher.

In some instances, the compositions described herein can be used to identify an unknown biological sample. A biological sample can be collected and contacted with a first and a second sub-plurality of primers corresponding to target sequences corresponding to regions of genomic DNA. With the aid of a computer processor, a first and a second data file can be separated to identify specific genomic abberations such as unique SNP's, which can be compared to a second sample in order to positively identify a biological sample. In some embodiments, the identification can be used for forensics applications. In some embodiments, the identification can be used for law enforcement applications. In some embodiments, the identification can be used for bioterrorism applications.

Methods provided herein can be used to prognose, diagnose, or monitor a condition, e.g., a disease, e.g., cancer, neurological disorder, or prenatal condition (e.g., aneuploidy). The conditions or cancers can include, for example, acute myeloid leukemia; bladder cancer, including upper tract tumors and urothelial carcinoma of the prostate; bone cancer, including chondrosarcoma, Ewing's sarcoma, and osteosarcoma; breast cancer, including noninvasive, invasive, phyllodes tumor, Paget's disease, and breast cancer during pregnancy; central nervous system cancers, adult low-grade infiltrative supratentorial astrocytoma/oligodendroglioma, adult intracranial ependymoma, anaplastic astrocytoma/anaplastic oligodendroglioma/glioblastoma multiforme, limited (1-3) metastatic lesions, multiple (>3) metastatic lesions, carcinomatous lymphomatous meningitis, nonimmunosuppressed primary CNS lymphoma, and metastatic spine tumors; cervical cancer; chronic myelogenous leukemia (CML); colon cancer, rectal cancer, anal carcinoma; esophageal cancer; gastric (stomach) cancer; head and neck cancers, including ethmoid sinus tumors, maxillary sinus tumors, salivary gland tumors, cancer of the lip, cancer of the oral cavity, cancer of the oropharynx, cancer of the hypopharynx, occult primary, cancer of the glottic larynx, cancer of the supraglottic larynx, cancer of the nasopharynx, and advanced head and neck cancer; hepatobiliary cancers, including hepatocellular carcinoma, gallbladder cancer, intrahepatic cholangiocarcinoma, and extrahepatic cholangiocarcinoma; Hodgkin disease/lymphoma; kidney cancer; melanoma; multiple myeloma, systemic light chain amyloidosis, Waldenstrom's macroglobulinemia; myelodysplastic syndromes; neuroendocrine tumors, including multiple endocrine neoplasia, type 1, multiple endocrine neoplasia, type 2, carcinoid tumors, islet cell tumors, pheochromocytoma, poorly differentiated/small cell/atypical lung carcinoids; Non-Hodgkin's Lymphomas, including chronic lymphocytic leukemia/small lymphocytic lymphoma, follicular lymphoma, marginal zone lymphoma, mantle cell lymphoma, diffuse large B-Cell lymphoma, Burkitt's lymphoma, lymphoblastic lymphoma, AIDS-Related B-Cell lymphoma, peripheral T-Cell lymphoma, and mycosis fungoides/Sezary Syndrome; non-melanoma skin cancers, including basal and squamous cell skin cancers, dermatofibrosarcoma protuberans, Merkel cell carcinoma; non-small cell lung cancer (NSCLC), including thymic malignancies; occult primary; ovarian cancer, including epithelial ovarian cancer, borderline epithelial ovarian cancer (Low Malignant Potential), and less common ovarian histologies; pancreatic adenocarcinoma; prostate cancer; small cell lung cancer and lung neuroendocrine tumors; soft tissue sarcoma, including soft-tissue extremity, retroperitoneal, intra-abdominal sarcoma, and desmoid; testicular cancer; thymic malignancies, including thyroid carcinoma, nodule evaluation, papillary carcinoma, follicular carcinoma, Hiirthle cell neoplasm, medullary carcinoma, and anaplastic carcinoma; uterine neoplasms, including endometrial cancer and uterine sarcoma.

Kits

Any of the compositions described herein may be comprised in a kit. In a non-limiting example, the kit, in a suitable container, comprises: a plurality of primers, wherein the plurality of primers comprises at least two sub-pluralities of primers. The kit can also comprise a computer readable medium, e.g., non-transitory computer readable medium, as described herein. The kit can also comprise reaction components for primer extension and amplification (e.g., dNTPs, polymerase, buffers). The kit can include reagents for library formation (e.g., primers (probes), dNTPs, polymerase, end repair enzymes). The kit may also comprise means for purification, such as a bead suspension. The kit can include reagents for sequencing, e.g., fluorescently labelled dNTPs, sequencing primers, etc.

The containers of the kits can include at least one vial, test tube, flask, bottle, syringe or other containers, into which a component may be placed and suitably aliquotted. Where there is more than one component in the kit, the kit also can contain a second, third or other additional container into which the additional components may be separately placed. However, various combinations of components may be comprised in a container.

When the components of the kit are provided in one or more liquid solutions, the liquid solution can be an aqueous solution. However, the components of the kit may be provided as dried powder(s). When reagents and/or components are provided as a dry powder, the powder can be reconstituted by the addition of a suitable solvent.

A kit can include instructions for employing the kit components as well the use of any other reagent not included in the kit. Instructions may include variations that can be implemented.

### EXAMPLES

### Example 1

### Primer Design and Synthesis

Four subsets of primers are designed to detect presence or absence of four classes of genomic alterations: abnormal gene expression (Subset 1), SNPs (Subset 2), alternative splicing events (Subset 3), and gene fusions (Subset 4). All primers include an Illumina adaptor sequence at a 5′ end and a target-specific sequence at a 3′ end.

Target-specific sequences of subset 1 primers are designed to reside within the two most 5′ and 3′ exons of genes suspected of having abnormal gene expression and optionally of genes suspected of having normal gene expression. For example, target-specific sequences of subset 1 primers also include sequences designed to reside within the two most 5′ and 3′ exons of ten housekeeping genes, which can be suspected of having normal gene expression. Subset 1 primers are further designed according to the following criteria: (1) primers have target-specific sequences designed to anneal to a genomic location entirely within an exon, or designed to span an exon-exon junction, (2) the target-specific sequence length of the primers are at least 35 bases in length, (3) primers have target-specific sequences designed to anneal to unique sequences within the transcriptomes, and (4) primers have target-specific sequences that are at least 25 bases from the exon junction. The sequences of the primers for detecting abnormal gene expression are recorded in a FASTA file labeled “expression”. The file can comprise target-specific sequences or the sequences of the entire primer, including the adaptor portions.

Target-specific sequences of subset 2 primers are designed such that 3′ ends of the primers are within 40 bases of a SNP. The sequences of these primers are recorded in a FASTA file labeled “SNP”.

Subset 3 primers comprise target-specific sequences which are designed to anneal to all reported exons within genes suspected of undergoing alternative splicing. The target-specific sequences of the primers are designed to be at least 40 bases in length. Such primers are also designed such that the 3′ end of each primer is between zero and about 25 bases from an exon junction. These primers are designed in each orientation relative to the exon junctions; e.g., they face the exons that are both 5′ and 3′ of the exon where the primer would anneal. The sequences of these primers are recorded in a FASTA file labeled “exon usage”.

Subset 4 primers for detection of gene fusion events can be designed according to the same principles as described for the subset 3 primers. For instance, subset 4 primers can comprise target-specific sequences which are designed to anneal to all reported exons within genes suspected of undergoing gene fusion events. The target-specific sequences of such primers can be designed to be at least 40 bases in length. Such primers can also be designed such that the 3′ end of each primer is between zero and about 25 bases from an exon junction. Such primers can also be designed in each orientation relative to the exon junctions; e.g., they face the exons that are both 5′ and 3′ of the exon where the primer would anneal. Subset 4 primers can include one or more primers in subset 3. Subset 4 primers can also include primers designed to genes previously implicated in fusion events. Exemplary genes which are previously implicated in fusion events include, but are not limited to ALK, BCR, and CDK6. To detect gene fusion events, primers used to monitor alternative splicing (exon usage FASTA file) and primers designed by the same criteria to genes previously implicated in fusion events with genes ALK, BCR and CDK6, are combined with the exon usage primers to ALK, BCR and CDK6 into a single FASTA file labeled “fusion”.

The primers corresponding to each class of genomic alterations are synthesized as one single pool of primers.

### Example 2

### Sample Preparation and Sequencing

Ovation Target Enrichment libraries are generated from 100 ng total RNA. Briefly, total RNA is converted into double stranded cDNA using NuGEN's cDNA module. Barcoded adapters containing a random hexamer tag (e.g., N6) to monitor fragment uniqueness are ligated onto these DNA molecules. The strands are then denatured. Primers described in Example 1 are annealed according to manufacturer's recommendations, and then extended. These libraries are enriched by PCR, using a separate set of primers designed to anneal to the adaptor sequences. The libraries are diluted to an appropriate concentration and sequenced on an Ilumina MiSeq. The sequencer is programmed to obtain 70 bases of the forward read (forward primer), a 14 base index read and 88 bases of a reverse read.

### Example 3

### Detecting Different Classes of Genomic Alterations

The resulting sequence data files are parsed for each sample by barcode, trimmed for quality, and duplicates removed. 18 bases are trimmed from the reverse read according to manufacturer's recommendations. The resulting data is further parsed by mapping the next 35 bases of the reverse read to each of the FASTA sequence files generated in primer design for measurement of abnormal expression, SNPs, alternative splicing events, and fusions. Each set of parsed reverse reads is paired with the corresponding set of forward reads. The resulting forward and reverse reads correspond to reads derived from the primers designed for each measurement and will be ready for independent analysis of gene expression, SNPs, alternative splicing and fusion detection.

Expression analysis is carried out by two independent methods. First, forward reads corresponding to the set of reads that mapped to the “expression” probe sequence file are aligned to the genome with RNA STAR. After alignment, Fragments Per Kilobase of transcript per Million mapped reads (FPKM) values for the targeted, as well as the housekeeping genes, are calculated using Cufflinks. Second, expression is calculated using the expression “probe counts,” or the number of times each “expression” probe sequence was present in the reverse read sequence. All values are normalized between replicates and across cell lines.

For SNP detection, the reverse reads corresponding to the set of reads that mapped to the “SNP” probe sequence file are aligned to the genome using BWA. Variants are detected using the GATK best practices pipeline.

For alternative splicing, the forward and reverse reads corresponding to the set of reads that mapped to the “exon usage” probe sequence file are aligned to the genome with RNA STAR. Exon usage/isoform expression is calculated using Cufflinks. Exon usage are dependent on having forward and reverse reads mapped to an exon present in a gene isoform, as well as reads spanning to neighboring exons across splice junctions.

For fusion detection, the forward and reverse reads corresponding to the set of reads that mapped to the “fusion” probe sequence file are analyzed by SOAPfuse. Fusion detection can be dependent on detecting instances where the forward and reverse reads came from different genes and where reads spanned splice junctions.

Alternatively, fusion detection is analyzed using a fusion analysis pipeline. As an optional first step, sequence reads are trimmed prior to analysis. Probes are then aligned to sequence reads using 8 base pair K-mers. The raw reads are then optionally deduplicated using an N6 sequence in the index read and the probe-assigned sequence reads are quality trimmed. The sequence reads are then aligned by STAR alignment, which is then optionally filtered to remove reads which do not fully contain an expected probe sequence. The aligned and optionally filtered sequence is then processed by STAR-Fusion, which can be filtered to post process called fusions based on expected contributing probes. [

For CNV detection, sequence reads that have been optionally trimmed to remove adaptor and poor quality reads are aligned using a Bowtie approach. The sequence reads can then be optionally deduplicated, and the number of forward reads that fall within a region of interest can be counted. The count is then normalized against the total number of sequence reads, and the gene level copy number level can be optionally determined by averaging normalized counts across the gene. These normalized counts can optionally be compared to the number of reads in a control sample.

While preferred embodiments have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments described herein may be employed in practicing the disclosure.

1. A method for detecting presence or absence of two or more classes of genomic alterations in a single assay, the method comprising:
(a) sequencing a plurality of polynucleotide library members to produce sequence reads;
(b) with aid of a computer processor, querying the sequence reads for presence of a sequence corresponding to any one of a first or second sub-plurality of a plurality of primers, wherein the first sub-plurality of primers comprises sequence designed to prime extension reactions into target sequence corresponding to genomic locations suspected of harboring a first class of genomic alterations and the second sub-plurality of primers comprises sequence designed to prime extension reactions into target sequence corresponding to genomic locations suspected of harboring a second class of genomic alterations, wherein the first class of genomic alterations and second class of genomic alterations are different, thereby identifying a first subset of sequence reads generated by sequencing the polynucleotide library members generated using the first sub-plurality of primers and a second subset of sequence reads generated by sequencing the polynucleotide library members generated using the second sub-plurality of primers;
(c) with aid of a computer processor, separating the first subset of sequence reads into a first data file, and separating the second subset of sequence reads into a second data file; and
(d) with aid of a computer processor, analyzing the first subset of sequence reads for presence or absence of the first class of genomic alterations, and analyzing the second subset of sequence reads for presence or absence of the second class of genomic alterations.

2. The method of claim 1, further comprising, before (a), hybridizing the plurality of primers to a sample of polynucleotides.

3. The method of claim 2, further comprising extending the plurality of primers with a polymerase, thereby generating polynucleotide extension products.

4. The method of claim 3, further comprising amplifying the polynucleotide extension products, thereby generating amplification products.

5. The method of claim 3, wherein the polynucleotide extension products are the polynucleotide library members of (a)

6. The method of claim 4, wherein the amplification products are the polynucleotide library members of (a).

7. The method of claim 1, wherein the plurality of primers comprises n additional sub-pluralities of the plurality of primers comprising target-specific sequences designed to extend into target sequence corresponding to genomic locations suspected of harboring n additional classes of genomic alterations.

8. The method of claim 7, wherein the sequence reads of (a) further comprise n additional subsets of sequence reads comprising sequences corresponding to the n additional sub-pluralities of the plurality of primers.

9.-58. (canceled)

59. A non-transitory computer readable medium comprising computer executable code for detecting presence or absence of two or more classes of genomic alterations in a sample subjected to a single assay, the computer readable medium comprising:
(a) a database comprising a set of oligonucleotide sequences corresponding to a set of primers, wherein the set of oligonucleotide sequences comprises:
(i) a first subset of oligonucleotide sequences corresponding to a first subset of primers, wherein the first subset of primers are designed to prime an extension reaction into target sequence corresponding to genomic locations suspected of harboring a first class of genomic alterations, and
(ii) a second subset of oligonucleotide sequences corresponding to a second subset of primers, wherein the second subset of primers are designed to prime an extension reaction into target sequence corresponding to genomic locations suspected of harboring a second class of genomic alterations;
(b) a set of computer executable instructions that, when executed by a processor, performs:
(i) receiving a set of sequence reads;
(ii) querying the set of sequence reads for presence of a sequence belonging to the first subset of oligonucleotide sequences or second subset of oligonucleotide sequences in the database;
(iii)transferring sequence reads which comprise a sequence belonging to the first subset of oligonucleotide sequences into a first data file;
(iv) transferring sequence reads which comprise a sequence belonging to the second subset of oligonucleotide sequences into a second data file; and
(v) analyzing the sequence reads transferred to the first data file for presence or absence of a first class of genomic alterations, and analyzing the sequence reads transferred to the second data file for presence or absence of a second class of genomic alterations.

60. The non-transitory computer readable medium of claim 59, wherein the set of oligonucleotide sequences further comprises n additional subsets of primers, wherein the n additional subsets of primers are designed to prime an extension reaction into target sequence corresponding to genomic locations suspected of harboring n additional classes of genomic alterations.

61. The non-transitory computer readable medium of claim 60, wherein the querying further comprises querying the set of sequence reads for presence of a sequence belonging to any one of the n additional subsets of oligonucleotide sequences in the database.

62. The non-transitory computer readable medium of claim 61, wherein (iv) further comprises transferring sequence reads which comprise a sequence belonging to at least one of the n additional subsets of oligonucleotide sequences into a corresponding nth additional data file.

63. The non-transitory computer readable medium of claim 62, wherein (v) further comprises analyzing the sequence reads transferred to the nth additional data files for presence or absence of an nth additional class of genomic alterations.

64. The non-transitory computer readable medium of claim 61, wherein the analyzing of (v) comprises simultaneously analyzing.

65. The non-transitory computer readable medium of claim 60, wherein at least one of the first class, second class, or n additional classes of genomic alterations are selected from the group consisting of single nucleotide polymorphisms (SNPs), insertions, deletions, alternative splicing events, gene fusion events, altered expression levels, copy number variations, copy number alterations, inversions, and translocations.

66.-74. (canceled)

75. A computer system for detecting presence or absence of two or more classes of genomic alterations in a sample subjected to a single targeted assay, comprising:
(a) a database comprising:
(i) a first subset of oligonucleotide sequences corresponding to a first subset of primers, wherein the first subset of primers are designed to prime an extension reaction into target sequence corresponding to genomic locations suspected of harboring a first class of genomic alterations, and
(ii) a second subset of oligonucleotide sequences corresponding to a second subset of primers, wherein the second subset of primers are designed to prime an extension reaction into target sequence corresponding to genomic locations suspected of harboring a second class of genomic alterations; and
(b) a receiver configured to receive a set of sequence reads generated by sequencing a plurality of polynucleotide library members, wherein the polynucleotide library members were extended using
(i) the first subset of primers, and
(ii) the second subset of primers;
and
(c) a processor operatively coupled to the receiver, wherein the processor comprises computer executable instructions that, when executed by the processor, performs:
(i) querying the set of sequence reads for presence of a sequence belonging to the first subset of oligonucleotide sequences or second subset of oligonucleotide sequences in the database;
(ii) transferring sequence reads which comprise a sequence belonging to the first subset of oligonucleotide sequences into a first data file;
(iii) transferring sequence reads which comprise a sequence belonging to the second subset of oligonucleotide sequences into a second data file;
(iv) analyzing the sequence reads transferred to the first data file for presence or absence of a first class of genomic alterations, and analyzing the sequence reads transferred to the second data file for presence or absence of a second class of genomic alterations.

76. The computer system of claim 75, wherein the single targeted assay is a single targeted sequencing assay.

77. The computer system of claim 75, wherein (c)(iv) comprises simultaneously analyzing the sequence reads transferred to the first data file and the sequence reads transferred to the second data file.

78. The computer system of claim 75, wherein the database is a data file or a list.

79. A kit for detecting presence or absence of two or more classes of genomic alterations in a sample subjected to a single targeted assay, comprising:
(a) a plurality of primers, wherein the plurality of primers comprises
(i) a first subset of primers designed to prime an extension reaction into target sequence corresponding to genomic locations suspected of harboring a first class of genomic alterations, and
(ii) a second subset of primers designed to prime an extension reaction into target sequence corresponding to genomic locations suspected of harboring a second class of genomic alterations, wherein the first class of genomic alterations and the second class of genomic alterations are different;
(b) a polymerase; and
(c) instructions for detecting presence or absence of two or more classes of genomic alterations in a single targeted assay.

80.-110. (canceled)
